(19) United States
(12) Patent Application Publication
(10) Pub. No.: US 2008/0160086 A1
(43) Pub. Date: Jul. 3, 2008

(54) Delivery Systems For Calcium

(75) Inventor: Jonathan Farber, Montreal (CA)

Correspondence Address: STERNE, KESSLER, GOLDSTEIN & FOX P.L.L.C., 1100 NEW YORK AVENUE, N.W., WASHINGTON, DC, 20005, US

(73) Assignee: Scepter Holdings, Inc., Scarbrough (CA)

(21) Appl. No.: 10/590,155

(22) PCT Filed: Oct. 29, 2004
(86) PCT No.: PCT/CA04/01894

§ 371 Date: Mar. 2, 2007

Related U.S. Application Data
(60) Provisional application No. 60/546,513, filed on Feb. 19, 2004.

(51) Int. Cl. A61K 33/06; A61K 9/00; A61P 3/02

(52) U.S. Cl. 424/488; 424/682

(57) ABSTRACT

Oral gel delivery systems for calcium are provided comprising an ingestible matrix within which a calcium source is substantially uniformly and completely dispersed. The delivery systems may optionally include one or more other functional ingredients, for example, functional ingredients that aid in the uptake and/or metabolism of calcium by the body and/or that complement or enhance the function of calcium within the body.

### CROSS REFERENCE TO RELATED APPLICATIONS

This application is a continuation-in-part of U.S. patent application Ser. No. 10/416,547, filed Mar. 25, 2003, which is a national stage of PCT application PCT/CA03/00411, filed Mar. 25, 2003. The aforesaid PCT application claims priority from U.S. Provisional Patent Application Ser. No. 60/372,438, filed Apr. 16, 2002. The contents of all of the aforementioned applications are hereby specifically incorporated by reference in their entirety.

### FIELD OF THE INVENTION

The present invention pertains to the field of oral delivery systems, in particular to a gel delivery system for calcium.

### BACKGROUND OF THE INVENTION

Nutritional and dietary supplements such as multi-vitamins and minerals have grown in popularity, as evidenced by the tremendous growth in the industry involved in their manufacture, production and distribution. Calcium is an abundant mineral in the mammalian body that plays an important role in many fundamental physiological processes. Calcium deficiency can have a number of adverse effects, notably in the structure, function and integrity of the skeletal system.

The importance of calcium supplementation is becoming increasingly apparent, not only for those with inadequate diets, including growing children for whom the ability to build strong bone is particularly important, but also for those who lose bone mass as part of the natural aging process. Primary osteoporosis is common in both post-menopausal women, who lose bone mass at an accelerated rate due to the hormonal changes associated with menopause, and elderly men due to the age-related loss of bone mass. Secondary osteoporosis is also known and results from an identifiable disease process or agent.

Calcium has been added to a number of foodstuffs as a dietary supplement. Some of the more common problems associated with adding calcium at high levels to food or beverages include the “chalky” taste and the solubility and/or bioavailability characteristics of certain calcium salts.

A number of different forms of calcium supplement have been described that attempt to circumvent the problems inherent with high calcium content supplements. Many of these are designed to be administered in conventional caplet or tablet form, or as powders to be added to foods or beverages. For example, U.S. Pat. No. 6,203,823 describes a calcium taurate complex for use as a calcium supplement that is described as having excellent calcium bioavailability. The complex may also be useful in the treatment of hypertension and is intended to be administered in the form of a pharmaceutical composition. U.S. Pat. No. 6,528,542 describes a calcium formate salt for administration in gelatine caplet or tablet form. The calcium formate salt is intended to reduce phosphate absorption in the intestine and also increase calcium intake. U.S. Pat. No. 6,582,722 describes a calcium picolinate chelate for addition to food or beverages that provides calcium with high bioavailability. U.S. Pat. No. 6,569,477 describes a method for processing calcium to provide a calcium salt in powder form that is highly soluble and stable and can be readily solubilised in aqueous solution. U.S. Patent Application 2003/0118695 describes an edible water-in-oil emulsion that comprises calcium sulphate.

Other nutritional factors, such as Vitamins D and K, and minerals such as magnesium and zinc, are known to play a role in bone formation and, therefore, are frequently included in supplements together with calcium. Such combined supplements have been described for use in improving for bone health. For example, European Patent No. EP 0 702 954 describes a supplement containing calcium, Vitamin D, boron, copper, magnesium, manganese and zinc, that can be in solid or powder form. U.S. Pat. Nos. 5,698,222 and 5,817,351 describe supplements containing calcium glycerophosphate and Vitamin D in either solid or liquid form. European Patent No. EP 0 583 378 describes supplements comprising calcium citrate malate and vitamin D, and optionally oestrogen. The supplements can be provided as beverages, in solid form, such as tablets or capsules, or as dried powders that can be reconstituted in beverages. U.S. Pat. No. 6,436,446 also describes calcium-fortified beverages comprising calcium and Vitamin D. These supplements further comprise soy isoflavones, inulin and magnesium. U.S. Patent Application 2004/0013743 describes specific formulations of vitamins and minerals, including calcium, with phytoestrogens suitable for use as dietary supplements for women at various life stages. The dietary supplements are described as being suitable for formulation into capsules, tablets, powders (for mixing with consumable liquids), gels or syrups (for mixing into dietary liquids or foods). Alternatively, they may be formulated with other foods or liquids to provide premeasured food bars.

As an alternative to providing calcium supplements in beverage or tablet form, some manufacturers have designed calcium supplements that are in the form of a food. For example, U.S. Patent Application 2004/0013743, discussed above, indicates that the described dietary supplements may be formulated with other foods or liquids to provide premeasured food bars. U.S. Pat. No. 6,576,253 describes a calcium-enriched food bar specifically formulated for pregnant or lactating women, or women attempting to become pregnant. The food bar contains calcium, docosahexaenoic acid (DHA) and one or more DHA taste-masking agents in a base of carbohydrate, protein and fat.

Calcium supplements in a candy-type chewable format, which may be more organoleptically acceptable to consumers, have also been described. U.S. Pat. No. 4,582,709, for example, describes a soft, nougat type candy containing calcium or other minerals. The nougat base is made by preparing a whipped component comprising a protein and combining this with a syrup component. An edible polyol is also included in amounts of no more than 5% by weight. Inclusion of more than 5% of the polyol resulted in a product that was difficult to process and had unpleasant organoleptic qualities. U.S. Pat. No. 6,482,465 describes a chewy nougat composition that comprises actives in which a fully functionalized mass is obtained without cooking. The nougat may contain at least 20% calcium by weight and a humectant. The working examples describe glycerine as humectant at a maximum of 3%. U.S. Pat. No. 6,077,557 describes a gelled food composition comprising calcium. The composition is prepared as a slurry gel base comprising sugars, generally fruit materials, and a gel-forming agent. Calcium is suspended in the slurry in an insoluble form and the final composition is formed and cured. The composition further comprises as an essential element, a calcium sequestering agent, that is necessary to bind any soluble calcium in the composition. European Patent Nos. EP 0 966 208 and EP 1 340 428, and U.S. Pat. No. 6,673,380, describe chewy confectionery type calcium supplement that is prepared by caramelising a basic mix of sugars and then adding calcium and optionally other vitamins and minerals. The basic mix comprises between 40% and 70% by weight of sugars and may also comprise a small amount of a hydrocolloid (0.01% to 0.2%). The final moisture content of the supplement is below 10%.

Other chewable delivery systems for minerals and other functional ingredients have been described. Troches (or lozenges), for example, are a traditional drug dosage format that is based on gelatine and glycerine and used in preparing custom medications by hand for individual patients. Troches are made in small quantities from a base that typically comprises 70% glycerine, 10% gelatine and 20% water. The water is slowly driven off by heating the base and the final composition, which tends to absorb moisture from the air, is stored under refrigeration. The troche itself is made by re-melting the base and adding milligram quantities of an active ingredient. Troches are not stable and are intended to be consumed within thirty days. Typically, methyl paraben is included in the base material to prevent microbial spoilage.

U.S. Pat. No. 4,882,154 describes a more shelf-stable gelatine-based chewable delivery system. This system, however, requires the use of pre-coated drugs, vitamins and minerals in order to preserve the stability of these compounds. International Patent Applications WO 03/026438, WO 03/26439 and WO 03/088755 describe gel-like delivery systems for creatine and other active ingredients. The delivery systems described by these latter applications comprise as essential components a carbohydrate (such as a starch) and at least one hydrocolloid component (such as gelatine or a plant gum).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a delivery system for calcium. In accordance with an aspect of the present invention, there is provided an oral gel delivery system for calcium comprising one or more sources of calcium substantially uniformly dispersed in a gel matrix, said delivery system having a final moisture content of between about 10% and about 40% by weight and a water activity of less than about 0.9, and said gel matrix comprising (a) one or more hydrocolloids; (b) one or more sugars, sugar syrups, sugar alcohols, or a combination thereof; and (c) one or more polyhydric alcohols.

In accordance with another aspect of the present invention, there is provided an oral gel delivery system for calcium comprising one or more sources of calcium substantially uniformly dispersed in a gel matrix, said delivery system having a final moisture content of between about 10% and about 30% by weight and a water activity of less than about 0.7, and said gel matrix comprising (a) one or more hydrocolloids selected from the group of: modified starch, gelatine, gellan and pectin; (b) one or more sugar syrups selected from the group of: corn syrup, high fructose corn syrup, maltitol syrup and isomalt syrup, and (c) one or more polyhydric alcohols selected from the group of: glycerol and propylene glycol.

In accordance with another aspect, the oral gel delivery system of the present invention further comprises one or more functional ingredients, wherein the total amount of said one or more sources of calcium and said one or more functional ingredients is less than or equal to 40% by weight of said delivery system.

In accordance with another aspect of the present invention, there is provided a use of a gel matrix comprising (a) one or more hydrocolloids; (b) one or more sugars, sugar syrups, sugar alcohols, or a combination thereof, and (c) one or more polyhydric alcohols, in the preparation of an oral gel delivery system for calcium, wherein said delivery system comprises one or more sources of calcium substantially uniformly dispersed in said gel matrix, and said delivery system has a final moisture content of between about 10% and about 40% by weight and a water activity of less than about 0.9.

In accordance with another aspect of the present invention, there is provided a process for preparing an oral gel delivery system for calcium, said process comprising the steps of (i) preparing a blend of one or more hydrocolloids, one or more sugars, sugar syrups, sugar alcohols, or a combination thereof, and optionally water at a temperature of less than 100° C., wherein said hydrocolloid(s), said sugars, sugar syrups and/or sugar alcohols and said water are in a ratio that will provide a final moisture content to the delivery system of between about 10% and about 40% by weight; (ii) reducing the temperature of said blend to between about 50° C. and about 80° C.; (iii) dispersing one or more sources of calcium in a solvent comprising one or more polyhydric alcohols at a temperature at or below about 70° C. to provide a solvent mixture; (iv) combining said blend from step (ii) with said solvent mixture to provide a gel matrix, and (v) moulding said gel matrix to provide said oral gel delivery system.

In accordance with another aspect of the present invention, there is provided an oral gel delivery system for calcium prepared by the above-described process.

In accordance with another aspect, there is provided a use of an effective amount of an oral gel delivery system of the invention as a calcium supplement for an animal in need thereof.

In accordance with another aspect, there is provided a kit for the delivery of calcium to an animal comprising one or more units of an oral gel delivery system of the invention and optionally instructions for use.

### BRIEF DESCRIPTION OF THE FIGURES

FIG. 1 demonstrates absorption of a functional ingredient into the blood following administration of a delivery system prepared with a gel matrix according to one embodiment of the invention.

### DETAILED DESCRIPTION OF THE INVENTION

Unless defined otherwise, all technical and scientific terms used herein have the same meaning as commonly understood by one of ordinary skill in the art to which this invention pertains. As used herein, percentage values (%) represent the weight percentages of the total weight of the delivery system.

The term “functional ingredient,” as used herein, includes physiologically or pharmacologically active substances intended for use in the treatment, prevention, diagnosis, cure or mitigation of disease or illness, or that provide some degree of nutritional, physiological or therapeutic benefit to an animal when consumed. The term refers more particularly to a substance that affects beneficially one or more target functions in the body, in a way that is either an improved state of health or well-being and/or reduction of risk of disease. Non-limiting examples include drugs, botanical extracts, enzymes, hormones, proteins, polypeptides, antigens, nutritional supplements such as fatty acids, antioxidants, vitamins, minerals, as well as other pharmaceutically or therapeutically useful compounds. A functional ingredient in the context of the present invention refers to an ingredient included in the delivery system of the invention in addition to those ingredients that constitute the gel matrix itself. In the context of the present invention, calcium is a functional ingredient.

The terms “source of calcium” or “calcium source,” as interchangeably used herein, refer to a source of calcium suitable for dietary consumption and include, but are not limited to, elemental calcium, various calcium salts and calcium chelates as well as naturally occurring substances that are rich in calcium, such as egg shells, oyster shells, milk solids, bone, microcrystalline hydroxyapatite complex (MCHC) and the like.

The term “nutritional supplement,” as used herein, refers to a substance that exerts a physiological effect on an animal. Typically, nutritional supplements fulfil a specific physiological function or promote the health or well-being of the consumer.

The term “drug,” as used herein, refers to a pharmacologically active substance that exerts a localised or systemic effect or effects on an animal.

The terms “botanical extract” and “botanical,” as used interchangeably herein, refer to a substance derived from a plant source. Non-limiting examples include echinacea, Siberian ginseng, ginko biloba, kola nut, goldenseal, golo kola, schizandra, elderberry, St. Johns Wort, valerian, ephedra and the like.

The terms “subject” and “patient” as used herein refer to an animal in need of treatment.

The term “animal,” as used herein, includes, but is not limited to, mammals (including humans), birds and reptiles.

As used herein, the term “about” refers to a +/−10% variation from the nominal value. It is to be understood that such a variation is always included in any given value provided herein, whether or not it is specifically referred to.

### Calcium Delivery Systems

The calcium delivery systems according to the present invention are oral gel delivery systems comprising one or more source of calcium dispersed in an ingestible matrix. The delivery system may optionally comprise one or more other functional ingredients. The matrix of the delivery system provides for substantially uniform and complete dispersion of the calcium source (and other functional ingredients) and helps to minimise degradation of heat labile functional ingredients during manufacturing. The matrix of the delivery system further provides for minimised degradation of the functional ingredients during subsequent storage of the final delivery system. The source(s) of calcium, or combinations of calcium and other functional ingredients, provided by the delivery systems are useful, for example to maintain or improve bone density in a mammal.

While the primary functional ingredient in the delivery system is calcium, the delivery system can be formulated to accommodate specific combinations of other functional ingredients that are selected as nutritional supplements and/or to provide a specific physiological effect, for example, weight loss, sexual health, cardiovascular health, joint health, bone health, anti-oxidant effects, appetite suppression, thermogenesis, memory enhancement, performance enhancement, digestive health, or to help prevent colds or fight infection. In one embodiment, the delivery system comprises one or more other functional ingredients that aid in the uptake and/or metabolism of calcium by the body and/or that complement or enhance the function of calcium within the body.

The delivery system of the present invention comprises one or more sources of calcium (and optionally other functional ingredients) substantially uniformly dispersed within a gel matrix which comprises 1) one or more hydrocolloids; 2) a sugar component and 3) a solvent component. The selection of appropriate hydrocolloid(s) as described herein in amounts within the ranges indicated results in a matrix that readily retains the solvent component and thereby helps to prevent separation of the solvent from other components of the matrix. Additives, such as natural or artificial flavourings, colourings, acidulants, buffers and sweeteners can be included in conventional amounts in the matrix. The matrix may also include one or more sources of monovalent cations or divalent cations (in addition to calcium), if required, to allow for proper set-up of the matrix. If insufficient water is provided by the various components selected to formulate the matrix, additional water may be added to the matrix as necessary to provide the desired final moisture content within the range indicated below.

The delivery system may further comprise one or more compounds that act to enhance the bioavailability of the calcium and other functional ingredients (i.e. “bioavailability enhancers”), as discussed in more detail below.

Due to the substantially uniform and complete dispersion of the calcium within the matrix, the delivery systems of the invention are suitable for division into sub-units. For example, if a single unit of a delivery system is divided into three subunits, each subunit will contain a third of the dose of the original unit. Such division would not be possible with other delivery systems in which the functional ingredients are not evenly dispersed.

As indicated above, the matrix of the delivery systems provides for minimised degradation of functional ingredients during the preparation of the matrix and the storage of the final delivery systems. The use of relatively low temperatures in the preparation of the matrix, when compared to typical manufacturing procedures for confectioneries, ensures that the functional ingredients are not degraded by excessive heat. In accordance with the present invention, the functional ingredients are added to the other components of the matrix to prepare the delivery system at a temperature of 100° C. or less. In one embodiment of the present invention, the entire preparation process takes place at or below 100° C. In another embodiment, the delivery systems are prepared at or below a temperature of 75° C. In another embodiment, the delivery systems are prepared at or below a temperature of 70° C. In a further embodiment, the delivery systems are prepared at or below a temperature of 65° C. Low temperatures can be employed in the preparation of the delivery system because the matrix is formulated to be flowable at low temperatures by selection of appropriate ingredients as described herein. In one embodiment of the invention, the matrix is flowable at or above 45° C. In another embodiment, the matrix is flowable at or above 35° C.

The delivery systems of the present invention are intermediate moisture products and maintain a low interaction with water during and after preparation of the matrix, which can also contribute to the stability of some of the functional ingredients dispersed therein. Although the actual amount of moisture and final water activity (a_{w}) of an intermediate moisture food has not been defined precisely, general opinion is that an intermediate moisture product should have a moisture content between about 10% and about 40% by weight and an a_{w} below about 0.9 (see, S. Hegenbart, “Exploring Dimensions in Intermediate Moisture Foods,” (1993) Food Product Design, Weeks Publishing Company, Northbrook, Ill.). In accordance with the present invention, therefore, the final moisture content of the delivery systems is between about 10% and about 40%. In one embodiment, the final moisture content of the delivery systems is between about 10% and about 30%. In another embodiment, the final moisture content of the delivery systems is between about 11% and about 25%. In other embodiments, the moisture content is between about 13% and about 20%, and between about 14% and about 18%.

In addition, the delivery systems of the present invention have an a_{w}, below about 0.9. In one embodiment of the invention, the water activity of the final delivery systems is below about 0.85. In another embodiment, the water activity of the final delivery systems is below about 0.8. In a further embodiment, the water activity is below about 0.7. In another embodiment, the water activity is below about 0.6. Alternatively, the water activity of the final delivery systems may be described as being between about 0.45 and about 0.7. In one embodiment, the water activity is between about 0.5 and about 0.6.

For those functional ingredients that are susceptible to degradation, for example, due to heat lability, degradation during the process of preparing the matrix of the delivery systems is minimised. In one embodiment, degradation of the functional ingredients during preparation of the matrix is less than about 20%. In another embodiment, degradation of the functional ingredients during preparation of the matrix is less than about 15%. In other embodiments, degradation of the functional ingredients during preparation is less than about 10%, less than about 5%, less than about 3% and less than about 2%.

Degradation of the functional ingredients during storage of the final delivery systems under normal storage conditions (i.e. at temperatures of 30° C. or below) is also minimised. In accordance with the present invention, therefore, degradation of the functional ingredients during storage of the delivery systems under normal conditions is less than about 20%. In one embodiment, degradation of the functional ingredients during storage is less than about 15%. In other embodiments, degradation of the functional ingredients during storage is less than about 10%, less than about 5%, less than about 3% and less than about 2%.

The matrix to be used in the delivery systems of the invention can be formulated to have a final pH in the range of about 2.5 to about 9.0. As will be appreciated by one skilled in the art, however, selection of the final pH for the matrix will be influenced by the properties of the functional ingredients to be included in the final delivery system. Thus, for the calcium delivery systems of the invention, the matrix is formulated such that the delivery systems have a final pH in the range of about 5.0 to about 9.0. In one embodiment, the matrix is formulated such that the delivery systems have a final pH in the range of about 5.5 to about 9.0. In another embodiment, the matrix is formulated such that the delivery systems have a final pH in the range of about 6.0 to about 9.0. In further embodiments, the matrix is formulated such that the delivery systems have a final pH in the range of about 6.0 to about 8.5 and about 6.5 to about 8.5.

In their final form, the delivery systems of the present invention are semi-solid, intermediate moisture systems, having some properties clearly identified with those of jellies and some properties that are similar to the jujube variety of confectioneries. In the context of the present invention, the term “semi-solid” indicates that the delivery system has properties that, depending on the measurement, are a mixture of solid and liquid behaviours. The matrix of the delivery systems, therefore, is formulated to be semi-solid at normal room temperature. In the event, however, that the matrix liquefies due to exposure to elevated temperatures, the formulation of the matrix is such that no phase separation of the components occurs and the matrix can be readily re-solidified by cooling (for example, by cooling to temperatures of around 4° C.). The reformed product maintains the substantially uniform dispersion of the calcium (and other optional functional ingredients) contained therein. In one embodiment of the present invention, the delivery systems are formulated such that the matrix is a semi-solid at temperatures at or below about 40° C. In another embodiment, the delivery systems are semi-solid at or below about 35° C. In other embodiments, the delivery systems are semi-solid at or below about 30° C. and at or below about 25° C.

The gel delivery systems according to the present invention are suitable for administration to both human and non-human animals. One skilled in the art will appreciate that each delivery system can be formulated differently according to the type of animal to which it is to be administered. For example, for administration to an animal such as a cat or a dog, meat or fish-based flavours may be added. For administration to a human, the delivery system may be formulated, for example, as a confectionery using fruit-based or other confectionery flavours. The delivery systems are especially suited for oral administration due to their palatability. Additionally, due to the highly portable format, the delivery systems are simple and convenient to administer and to consume for both humans and other animals.

The texture, physical attributes, form and shape of the matrix as described below, can be varied by altering the ratio of ingredients within the given ranges using the methods described herein or by methods familiar to a worker skilled in the art.

### 1. The Matrix

As indicated above, the delivery systems of the invention comprise one or more sources of calcium dispersed in a matrix that comprises 1) one or more hydrocolloids; 2) a sugar component and 3) a solvent component. For the purposes of the present invention, “hydrocolloids” can be divided into carbohydrate-based hydrocolloids and non-carbohydrate based hydrocolloids. The delivery system of the present invention can comprise one or more carbohydrate-based hydrocolloids, one or more non-carbohydrate based hydrocolloids, or a combination of one or more carbohydrate-based hydrocolloids with one or more non-carbohydrate based hydrocolloids.

### 1.1 Hydrocolloid

The matrix according to the present invention comprises one or more hydrocolloids that perform the functions of water binding and gelation and contribute to the overall texture and body of the gel matrix. Hydrocolloids can also be used to improve and/or stabilise the texture of a food product while inhibiting crystallisation.

Hydrocolloids are hydrophilic polymers of vegetable, animal, microbial or synthetic origin. Non-carbohydrate based hydrocolloids are typically animal-derived, a representative example being gelatine (hydrolysed collagen). Carbohydrate-based hydrocolloids are typically plant derived and include starches (and other amylaceous ingredients) and polysaccharide-based gums. An “amylaceous ingredient” as used herein refers to a food-stuff that contains a preponderance of starch and/or starch-like material. Examples of amylaceous ingredients include cereal grains and meals or flours obtained upon grinding cereal grains such as corn, oats, wheat, milo, barley, rice, as well as the various milling by-products of these cereal grains such as wheat feed flour, wheat middlings, mixed feed, wheat shorts, wheat red dog, oat groats, hominy feed, and other such material. Other sources of amylaceous ingredients include tuberous foodstuffs, such as potatoes, tapioca, and the like.

Suitable starches for use in the delivery systems are typically modified starches derived from a variety of plant sources such as, for example, corn, waxy corn, wheat, rice, tapioca, potato, pea and other sources known in the art. Modified starches are known in the art refer to starches that have been physically or chemically altered to improve their bioactive characteristics. Suitable modified starches include, but are not limited to, pre-gelatinized starches, low viscosity starches (such as dextrins, acid-modified starches, oxidized starches and enzyme modified starches), derivatised starches, stabilised starches (such as starch esters and starch ethers), cross-linked starches, starch sugars (such as glucose syrup, dextrose and isoglucose) and starches that have been submitted to a combination of treatments (such as cross-linking and gelatinisation) and mixtures thereof.

Examples of suitable polysaccharide-based gums that can be used in the delivery systems include, but are not limited to, Konjac, tragacanth gum, guar gum, acacia gum, karaya gum, locust bean gum, xanthan gum, agar, pectin, carageenan, gellan, alginate, and various cellulose gums. Suitable cellulose gums for use in the preparation of the matrix are typically modified cellulose gums including, for example, methylcellulose (MC), hydroxypropyl methylcellulose (HPMC), ethyl cellulose (EC), hydroxyethyl cellulose (HEC), hydroxypropylcellulose (HPC), hydroxypropyl methylcellulose acetate, hydroxyethyl methylcellulose, hydroxyethylcellulose acetate, hydroxyethyl ethylcellulose and combinations thereof.

The use of hydrocolloids is well-known in the art and many hydrocolloids for use in products for human or animal consumption are available commercially, for example, gelatines from Leiner Davis, various polysaccharide gums and blends manufactured by CP Kelco, the Ticagel® range of hydrocolloids from TIC Gums, modified starches from A. E. Staley and a range of modified celluloses known as Methocel Food Gums manufactured by Dow Chemical Company.

In one embodiment of the present invention, the gel matrix comprises gelatine. Gelatine is defined generally using a “Bloom value” which indicates the strength of the gel formed under certain circumstances using the gelatine. In the preparation of confectionery, when a harder gel is desired, gelatine having a higher Bloom value is used. Conversely, when the final product is required to be more flowing, gelatine having a lower Bloom value is used. One skilled in the art will appreciate that the water holding capacity of gelatine alone is lower than that of a combination of gelatine with another hydrocolloid, such as gellan or pectin. Thus, the use of gelatine alone as the hydrocolloid in the delivery system may necessitate the use of a higher amount of gelatine to achieve the desired gelation/texture of the matrix, than when gelatine is used in combination with one or more other hydrocolloids. When the hydrocolloid in the matrix of the present invention comprises gelatine, the Bloom value (BL) is generally about 100 to 260 BL. Combinations of gelatines with different Bloom values also can be used. The gelatine can be derived from a variety of sources, for example, beef, pork, chicken or fish gelatine (or a combination thereof) may be used.

When the gel matrix comprises gelatine, the gelatine can be combined with one or more other hydrocolloids to impart different characteristics to the matrix. For example, combinations of gelatine with gellan or gelatine with pectin provide a good texture to the matrix. Addition of a modified starch to one of these combinations also provides textural improvements.

When combinations of gelatine and gellan or pectin are used in the preparation of the matrix, the ratio of gelatine:gellan or gelatine:pectin is typically in the range between about 15:1 to about 40:1. These relative amounts provide a cohesive structure to the delivery system.

Similarly, a combination of a modified starch with one or more other hydrocolloids can impart certain desirable features to the matrix, for example, modified starch can contribute to the structural integrity of the matrix and its low set temperature. It can also provide heat stability to the finished product as well as the ability to bind a limited quantity of fats/oils if required.

The use of combinations of modified starches and modified celluloses as the hydrocolloid component of the matrix is also contemplated by the present invention as discussed below in Section 1.5.

An example of a suitable type of modified starch for inclusion in the matrix is one that is able to fully hydrate and develop its viscosity in the presence of the other matrix-forming components at a temperature below 100° C., for example at a temperature of, or below, 70° C. Such starches are often referred to as “low set temperature” starches. While the majority of carbohydrates hydrate upon heating, certain starches, which are commercially available and are known in the art as “cold set” or “pre-gelatinised” starches are capable of hydrating at room temperature and are also suitable for use in the gel matrix.

One skilled in the art will appreciate that the viscosity development of the selected hydrocolloid or hydrocolloid mixture should allow for sufficient ease of mechanical handling and pumping during production as well as allowing sufficient time to incorporate all the ingredients and to mould the final product before it sets.

In addition, it will be understood that the hydrocolloid(s) to be used in the gel matrix will depend on the desired final pH of the matrix, the particular texture and consistency required for the final product and, if more than one hydrocolloid is used, the interaction of the hydrocolloids. Certain combinations of hydrocolloids are known in the art to provide synergistic effects, for example, the combination of xanthan (which does not gel well alone) with Konjac, or carageenan and Konjac.

The type of hydrocolloid, or mixture of hydrocolloids, used can also affect the set temperature of the matrix. For example, the use of a gelatine/gellan mixture or a gelatine/pectin mixture provides a set temperature around 35° C., whereas the use of carageenan or locust bean gum will result in a set temperature closer to 60° C. Thus, the choice of hydrocolloid(s) for use in the matrix is also dependent upon the properties of the functional ingredient(s) to be incorporated into the delivery system. Functional ingredients that are unstable at higher temperatures will require the selection of a hydrocolloid or mixture of hydrocolloids that have a low set temperature, whereas functional ingredients that are more stable can be used with hydrocolloid(s) having a higher set temperature.

The use of hydrocolloids in intermediate moisture products is well known in the art and a skilled technician would readily be able to select an appropriate hydrocolloid or mixture of hydrocolloids for use in the delivery systems of the invention. In one embodiment of the present invention, the delivery system comprises one or more modified starch, alone or in combination with one or more other hydrocolloid. Non-limiting examples of hydrocolloids suitable for use with modified starch include gelatine; gellan and gelatine; pectin and gelatine; gellan, gelatine and one or more cellulose or modified cellulose; and pectin, gelatine and one or more cellulose or modified cellulose. In another embodiment of the present invention, the delivery system comprises gelatine, alone or in combination with one or more other hydrocolloid. Non-limiting examples of hydrocolloids suitable for use with gelatine include one or more modified starch; gellan; pectin; cellulose or modified cellulose; gellan and one or more modified starch; pectin and one or more modified starch; gellan and one or more cellulose or modified cellulose; pectin and one or more cellulose or modified cellulose; gellan, one or more modified starch and one or more cellulose or modified cellulose; and pectin, one or more modified starch and one or more cellulose or modified cellulose. In a further embodiment of the present invention, the delivery system comprises pectin in combination with one or more other hydrocolloid. Non-limiting examples of hydrocolloids suitable for use with pectin include gelatine; gelatine and one or more modified starch; gelatine and one or more cellulose or modified cellulose; and gelatine, one or more modified starch and one or more cellulose or modified cellulose.

The total amount of hydrocolloid(s) incorporated into the matrix is generally between about 0.1% and about 17% by weight. In one embodiment, the total amount of hydrocolloid(s) in the matrix is between about 0.6% to about 17% by weight. In a further embodiment, the total amount is between about 0.6% and about 15% by weight. In another embodiment, the total amount is between about 0.5% and about 10% by weight.

The selection of the actual amount of hydrocolloid(s) from within the ranges provided above to be included in the matrix will be dependent upon the type of hydrocolloid(s) being used and on the desired texture of the final product. Determination of this amount is considered to be within the ordinary skills of a worker in the art.

In one embodiment of the invention, the matrix comprises one or more modified starch in an amount between about 0.5% and about 10.0% by weight, for example, between about 1.7% and about 8.0%. In another embodiment, the matrix comprises gelatine in an amount between about 0.1% and about 10% by weight, for example between about 1.0% and 9.0%. In a further embodiment, the matrix comprises a polysaccharide-based gum in an amount between about 0.1% and about 5.0% by weight, for example, between about 0.2% and about 2.0%. In still another embodiment, the matrix comprises one or more modified cellulose in an amount between about 0.1% and about 3% by weight, for example, between about 0.6% and 1.5%.

In a specific embodiment of the invention, the matrix comprises a combination of one or more modified starch in an amount between about 0.5% and about 10.0% by weight, gelatine in an amount between about 0.1% and about 10.0% by weight, and a polysaccharide-based gum in an amount between about 0.1% and about 2.0% by weight.

### 1.2 Sugar Component

Sugar is generally used in a confection primarily for sweetness; however, it is known in the art that sugar can also play an important role in the physical properties of a matrix, such as crystallinity, gel strength, bodying/texture, humectancy, and water activity.

The sugar component of the matrix comprises one or more sugars, sugar syrups, sugar alcohols and/or sugar alcohol solids. Examples include, but are not limited to, sugars such as sucrose, glucose, xylose, ribose, maltose, galactose, dextrose, and fructose; syrups such as corn syrups, hydrogenated glucose syrups, high fructose corn syrups; polydextrose; and sugar alcohols such as isomalt, maltitol, sorbitol, lactitol and mannitol. The latter are also often in the form of syrups. One skilled in the art will appreciate that if a sugar or sugar alcohol solid is used in the matrix, it should be first dissolved, for example, by heating in water or in another syrup, prior to being added to the mixture.

When the sugar component comprises dextrose, it is generally provided in the form of a corn syrup. Corn syrups are prepared by hydrolysis of starch and are characterised by dextrose equivalent (D.E.) values such that they are classified as low, medium or high D.E. syrups, with high D.E. syrups having a high concentration of dextrose and low D.E. syrups having a low concentration of dextrose. In one embodiment of the present invention, the sugar component used in the preparation of the matrix comprises a corn syrup and/or a high fructose corn syrup. Suitable corn syrups are typically those with a D.E. between 20 D.E. and 99 D.E., for example, between about 40 D.E. and 70 D.E.

Various corn syrups are commercially available. For example, 62 D.E. 1600 Corn Syrup (Casco Inc./ Canada Starch Operating Co. Inc.), SWEETOSE 4300 corn syrup (a 63 D. E. corn syrup; A. E. Staley Manufacturing Company; Decatur, Ill.) and Clearsweet® 63/43 IX corn syrup (a 63 D. E. corn syrup; Cargill/North America Sweeteners).

Combinations of sugars or sugar syrups are also suitable for use in the preparation of the matrix. Examples of suitable combinations of syrups include, but are not limited to, isomalt syrup and high fructose corn syrup, a high D.E. corn syrup and high fructose corn syrup and maltitol syrup and high fructose corn syrup.

One skilled in the art will appreciate that the total amount of the sugar component in the matrix will vary depending upon the type(s) of sugar used. For example, when sugar syrups are used, lower viscosity sugar syrups will produce a matrix with less body and lower rigidity. The total amount of the sugar component present in the matrix is about 10% to about 60% by weight.

In one embodiment of the present invention, the sugar component comprises a mixture of sugar syrups. In another embodiment, the sugar component comprises a mixture of sugar syrups in a total amount of between about 15% and about 55% by weight of the delivery system. In a further embodiment, the sugar component comprises a mixture of sugar syrups in a total amount between about 25% and about 55% by weight of the delivery system.

### 1.3 Solvent Component

The primary role of the solvent component of the matrix is to dissolve or disperse the functional ingredients to allow for substantially uniform and complete incorporation of these ingredients into the matrix. The solvent also provides for improved flow characteristics of the mixture and functions somewhat as a humectant. In accordance with one embodiment of the present invention, the calcium and/or other functional ingredients are added to the solvent component prior to combining with the remaining components of the matrix.

The solvent used in the preparation of the matrix is typically colourless and non-volatile with no strong odour or flavour and is substantially miscible with water and/or alcohols. In accordance with the present invention, the solvent component is one or more polyhydric alcohol. The term “polyhydric” as used herein means that the compound contains two or more hydroxyl groups. Examples of suitable polyhydric alcohols include, but are not limited to, glycerol and/or its lower alkyl ester derivatives, propylene glycol, and short chain polyalkylene glycols, such as polyethylene glycol, and mixtures thereof. As will be apparent to one skilled in the art, certain polyhydric alcohols may also function somewhat as sweeteners.

In one embodiment of the present invention, the solvent component comprises glycerol. In another embodiment, the solvent component comprises a mixture of glycerol and a short chain polyalkylene glycol. In a further embodiment, the solvent component comprises a mixture of glycerol and propylene glycol.

Typically, the delivery system according to the present invention contains about 5% to about 50% by weight of the solvent component. In one embodiment, the delivery system contains about 5% to about 35% by weight of the solvent component. In an alternate embodiment, the delivery system contains about 10% to about 50% by weight of the solvent component. In a further embodiment, the delivery system contains about 20% to about 48% by weight of the solvent component. In other embodiments, the delivery system contains between about 15% and about 50%, between about 15% and about 40% and between about 15% and 35% by weight of the solvent component.

### 1.4 Water

As indicated above, the delivery system according to the present invention has a final moisture content between about 10% and about 40% and a water activity below about 0.9. In one embodiment, the final moisture content of the delivery system is between about 10% and about 30% and the water activity is below about 0.7. It will be readily apparent to one skilled in the art that the appropriate amount of water may be provided by one or more of the various components of the system, for example, a sugar syrup, a hydrated starch or a hydrated hydrocolloid, or additional water may need to be added separately. Additional water can be provided alone or as a solution containing other additives, for example, as a buffer solution or as a solution containing a sweetener, flavouring or colouring. The total amount of water from the one or more sources will be sufficient to provide the final delivery system with a moisture content and water activity within the ranges indicated above.

### 1.5 Other Additives

The gel matrix can optionally contain other additives such as flavourings, colourings, additional sweeteners, modified vegetable gums or celluloses, mono- or divalent cations, or a combination thereof. It will be readily apparent that additives for inclusion in the matrix should be selected such that they do not affect the properties of the matrix, do not exhibit substantial reactivity with the functional ingredients in the matrix, and are stable during preparation of the matrix.

One or more additional sweeteners can be selected from a wide variety of suitable materials known in the art. Representative, but non-limiting, examples of sweeteners include xylose, ribose, sucrose, mannose, galactose, fructose, dextrose, maltose, partially hydrolysed starch, lactose, maltodextrins, hydrogenated starch hydrolysate and mixtures thereof. In addition to these sweeteners, polyhydric alcohols such as sorbitol, mannitol, xylitol, and the like may also be incorporated. Alternatively, an artificial sweetener or a blend of artificial sweeteners can be used. Examples of suitable artificial sweeteners include, for example, sucrose derivatives (such as Sucralose), amino acid based sweeteners, dipeptide sweeteners, saccharin and salts thereof, acesulfame salts (such as acesulfame potassium), cyclamates, steviosides, dihydrochalcone compounds, thaumatin (talin), glycyrrhizin, aspartame, neotame, alitame, and mixtures thereof. In one embodiment of the invention, the matrix comprises one or more additional sweeteners. In another embodiment, the matrix comprises one or more artificial sweeteners.

When an additional sweetener is used, it can be used in amounts as low as 0.01% by weight. The actual amount of sweetener required will be dependent on the type of sweetener selected and on the desired sweetness of the final product. Amounts of various sweeteners to be added to food products are well known in the art. When a natural sweetener is used, the total amount of the sugar component, which forms a structural part of the matrix, and additional sweetener(s) in the matrix, however, remains less than 60% by weight.

Suitable flavourings that can be added to the delivery system are known in the art and include, both synthetic flavour oils and oils derived from various sources, such as plants, leaves, flowers, fruits, nuts, and the like. Representative flavour oils include spearmint oil, peppermint oil, cinnamon oil, and oil of wintergreen (methylsalicylate). Other useful oils include, for example, artificial, natural or synthetic fruit flavors such as citrus oils including lemon, orange, grape, lime and grapefruit, and fruit essences including apple, strawberry, cherry, pineapple, banana, raspberry and others that are familiar to a worker skilled in the art. A wide variety of synthetic flavourings suitable for inclusion in the matrix are known in the art and are commercially available. The amount of flavouring agent employed is normally a matter of preference subject to such factors as concentration/dilution of the flavour stock, flavour type, base type and strength desired. In general, amounts of about 0.01% to about 5.0% by weight of a final product are useful.

Colourings suitable for use in foodstuffs are well known in the art and can be optionally included in the matrix to add aesthetic appeal. A wide variety of suitable food colorings are available commercially, for example, from Warner Jenkins, St. Louis, Mo. Where a synthetic colouring agent is used in the matrix, the amount ranges from about 0.01% to about 2% by weight. A worker skilled in the art will appreciate that when a colouring agent derived from a natural source is used in the matrix, an increased amount of the colouring agent is generally required to achieve the same effect as a synthetic colouring agent.

The present invention also contemplates that modified vegetable gums or modified or unmodified celluloses may be included in the matrix in order to improve the texture, body, lubricity and/or elasticity of the matrix. These compounds can be used, for example, to increase the viscosity of the delivery system if it is warmed, thus reducing potential melting and lessening water activity which will help to improve the stability of the system in the event it is left in an excessively hot environment. Examples of modified vegetable gums or modified celluloses are provided above. Unmodified celluloses are also contemplated and are known in the art. Examples include Solka-Flo® (International Fibre Corporation, North Tonawanda, N.Y.) and powdered Avicel® microcrystalline cellulose (FMC Biopolymers, Philadelphia, Pa.). Modified vegetable gums can be included in the matrix in amounts between about 0.01% and 2.0% by weight, for example between about 0.1% and about 1.5%. Modified or unmodified celluloses, or mixtures thereof, can be included in the matrix in amounts between about 0.1% and about 10.0% by weight, for example, between about 0.6% and about 5.0%.

If necessary, the matrix can also comprise one or more sources of monovalent cations and/or divalent cations (in addition to calcium) to help facilitate gelation of the matrix. Suitable sources of mono- and divalent cations for incorporation into food products are known in the art and are commercially available. Non-limiting examples include mono- or divalent salts, such as sodium or potassium chloride and potassium citrate. Mono- or divalent salts can be added to the matrix, if required, in an amount between, for example, about 1% and about 5% by weight.

### 2. Calcium Source

A number of calcium sources can be used to provide the calcium content of the delivery system. Examples of acceptable calcium sources include, but are not limited to, the following calcium salts: calcium ascorbate, calcium aspartate, calcium carbonate, calcium chloride, calcium citrate, calcium fumarate, calcium gluceptate, calcium gluconate, calcium glycerophosphate, calcium hydroxide, calcium hypophosphate, calcium lactate, calcium levulinate, calcium malate, calcium oxide, calcium pantothenate, calcium phosphate (including mono-, di- and tricalcium phosphate), calcium pyrophosphate, calcium pyruvate, calcium sulphate, calcium tartrate, tricalcium citrate tetrahydrate, D-gluconic acid (hemicalcium salt), and mixtures thereof. Calcium chelates and calcium amino acid chelates are also suitable sources of calcium. Many of these calcium sources are commercially available, for example, amino acid chelates, calcium aspartate, calcium malate, calcium citrate malate are available from Albion Advanced Nutrition (St. Clair Shores, Mich.), calcium phosphate is available from Garuda International (Lemon Cove, Calif.), calcium carbonate is available from Specialty Minerals Inc. (Bethlehem, Pa.), calcium lactate and calcium gluconate are available from PURAC America Inc. (Lincolnshire, Ill.) and calcium pyruvate is available from Watson Industries (City of Industry, Calif.).

Other salts of calcium have been described and constitute suitable calcium sources for the purposes of the present invention. These include, for example, calcium taurate (see U.S. Pat. No. 6,203,823), calcium formate (see U.S. Pat. No. 6,528,542), a combined calcium citrate malate (CCM) salt that has been described as having approximately six-times the solubility of either calcium citrate or calcium malate (Smith et al., Calcified Tissue International, 41: 351-352 (1987); International Patent Application WO 91/19692) and a calcium picolinate chelate, which demonstrates high bioavailability in humans (see, U.S. Pat. No. 6,582,722).

Additionally, calcium salts from reaction between an acid and calcium hydroxide may also be used. A method of making calcium salts by combining calcium carbonate or calcium hydroxide, and optionally another mineral hydroxide, with lactic acid, acetic acid, citric acid, malic acid, phosphoric acid, ascorbic acid, or other food grade acid is described in U.S. Pat. No. 6,582,722. The resulting salts are described as providing soluble bioavailable calcium and/or other minerals at a high concentration due to the high solubility attained through the processing method. Combined calcium/magnesium salts can also be made by this method that would be useful in the delivery systems of the present invention.

Natural sources of calcium are also contemplated, such as egg shell, oyster shell, limestone, milk calcium, bone, and mixtures thereof. Microcrystalline hydroxyapatite complex (MCHC) is a mixed mineral calcium source derived from bone that is a useful calcium source in the context of the present invention. MCHC can also provide other organic and inorganic nutrients to support bone health, including the natural elements present in healthy bone such as microcrystalline hydroxyapatite (crystalline calcium and phosphorus), collagen, bone amino acids, glycosaminoglycans and a range of bone trace minerals. MCHC is commercially available, for example, from Waitaki Biosciences International Ltd. (Christchurch, New Zealand).

Pre-processing of the calcium source by micronisation or pre-coating is not required for the delivery systems of the present invention, however, if desired, micronised or pre-coated calcium sources can be employed. Various coatings are known in the art that would be suitable for the purposes of the present invention (see, for example, U.S. Pat. No. 4,882,154). Micronisation of minerals is also standard in the art.

Up to 40% by weight of the selected calcium source(s) can be included in the delivery system of the invention. In one embodiment, up to about 35% by weight of the selected calcium source(s) is included in the delivery system. In another embodiment, up to about 30% by weight of the selected calcium source(s) is included. In an alternate embodiment, the delivery systems are formulated with about 10% to about 30% by weight of the calcium source(s). In a further embodiment, the delivery systems are formulated with about 10% to about 25% by weight of the calcium source(s).

### 3. Other Functional Ingredients

The present invention provides for the addition of one or more other functional ingredients to the calcium delivery system. A wide variety of functional ingredients are suitable for inclusion in the delivery system, including, but not limited to, therapeutic compounds, nutritional supplements that fulfil a specific physiologic function or promote the health and/or well-being of the consumer, botanicals, herbal extracts, and the like. For example, the one or more functional ingredients included in the delivery system can be a nutritional supplement. Illustrative, but non-limiting, examples of suitable nutritional supplements include, probiotic bacteria, prebiotics, vitamins, enzymes, co-enzymes, cofactors, antioxidants, minerals and mineral salts, amino-acids and amino acid derivatives (for example, dimethylglycine), peptides, proteins, gums, carbohydrates, phytochemicals, dextroses, phospholipids, other trace nutrients, oxygenators, brain-stimulating substances, energy providers, metabolic intermediates, hormones, botanical extracts, fatty acids (for example, linoleic acid or conjugated linoleic acid), oat beta-glucan and other functional fibres, carnitine, bicarbonate and citrate.

The selection of appropriate and compatible combinations of functional ingredients can be made readily by the skilled technician. As is known in the art, certain combinations of functional ingredients are incompatible due to undesirable interactions between the ingredients, for example, interactions that alter absorption, renal elimination, or hepatic metabolism of one or more of the functional ingredients, or that result in additive effects or toxicities. Accordingly, selection of appropriate combinations of functional ingredients can be made by the skilled worker based on knowledge in the art and publicly available information regarding contraindications of certain combinations (see, for example, The A-Z Guide to Drug-Herb and Vitamin Interactions, Schuyler W. Lininger (ed.) (1999) Three Rivers Press (CA); Mosby's Handbook of Drug-Herb & Drug-Supplement Interactions, R. Harkness & S. Bratman (2002), Mosby; and the Mayo Clinic website).

In one embodiment of the present invention, combinations of functional ingredients that are designed to achieve a specific physiological purpose are contemplated for inclusion in the delivery system. A wide variety of such combinations of functional ingredients are known in the art for providing specific physiological benefits and are suitable for inclusion in a calcium delivery system of the invention. Non-limiting examples are provided in Table 1.

TABLE 1
Exemplary Functional Ingredients and Combinations Thereof

| Physiological Effect | Suggested Functional Ingredients¹ |
|---|---|
| Energy enhancement | Ginseng, chromium picolinate, chromium chelate, |
| | Rhodiola crenulata |
| Weight loss | Caffeine, ephedra, conjugated linoleic acids |
| | (CLA), amino acids |
| Thermogenesis | Caffeine, tocopherols, Citrus aurantium, ephedra |
| | alkaloids, |
| Memory enhancement | Ginkgo biloba, goto kola |
| Sexual health | Yohimbe, Kubu pepper |
| Antioxidant | Vitamin E, Vitamin C, Alpha Lipoic Acid (ALA) |
| Bone health | Inulin, fructooligosaccharides, Vitamin D, |
| | Vitamin K, Vitamin C, magnesium, phosphorus, |
| | zinc, copper, boron, manganese, selenium, |
| | fluoride, isoflavones |
| Joint health | Methylsulphonylmethane (MSM), glucosamine, |
| | chondroitin |
| Cold prevention | Echinacea, zinc, vitamin C |
| Vitamin and/or | B-Vitamin complex, D vitamins, Vitamin C, |
| mineral | Vitamin co-factors |
| supplementation | |
| Dietary supplements | Essential fatty acids, amino acids |
| Muscle enhancement | Creatine, dimethylglycine, pregnenolone, |
| | amino acids |
| Probiotics | Acidiphilus, Bifidus, prebiotics, |
| Digestive aids | Bromelain, papain, lipases, probiotics, |
| Anti-aging | Omega-3 fatty acids, lignan, S-adenosyl |
| | methionine(SAMe), melatonin |
| Seniors health | Omega-3 fatty acids, SAMe |
| Women's health | Soy isoflavanones |
| Cardiovascular health | Arginine, Siberian Ginseng, Vitamin B6, CoQ10, |
| | Rhodiola crenulata |

¹Delivery systems may contain one, or a combination, of the listed functional ingredients

In one embodiment of the present invention, the delivery system comprises one or more additional functional ingredients that aids in the uptake and/or metabolism of calcium by the body and/or that complements or enhances the function of calcium within the body. The present invention also contemplates the inclusion of functional ingredients known to contribute to bone health in the delivery system. Examples of such functional ingredients include, but are not limited to, prebiotics, vitamins, antioxidants, minerals and mineral salts, amino-acids and amino acid derivatives, phytochemicals, hormones, botanical extracts, oat beta-glucan or other functional fibres, or combinations thereof.

Prebiotics can be delivered together with the calcium source in the delivery system. Prebiotics comprise carbohydrates, generally oligosaccharides, and have the ability to resist hydrolysis by enzymes in the animal digestive tract. Oligosaccharides may be produced from glucose, galactose, xylose, maltose, sucrose, lactose, starch, xylan, hemicellulose, inulin, or a mixture thereof. Purified commercially available products such as fructooligosaccharides and glucooligosaccharides often contain greater than about 95% solids in the form of oligosaccharides. Prebiotics often comprise a mixture of fructooligosaccharide (or oligofructose) and inulin, for example, PREBI01® or a mixture of commercially available RAFTILOSE® and RAFTILINE® commercialized by Orafti (Tienen, Belgium). Inulin is commercially available, for example, from Imperial Suiker Unier (Sugar Land, Tex.).

Intake of inulin and fructooligosaccharide in the diet or as a dietary supplement has been shown to provide a number of health benefits including increased mineral absorption, in particular calcium absorption (see review by Kaur & Gupta, J. Biosci., (2002), 27:703-714).

Other vitamins and minerals can be included in the delivery system. Examples include, but are not limited to, Vitamin D, Vitamin K, Vitamin C, magnesium, phosphorus, zinc, copper, boron, manganese, and combinations thereof.

Vitamin D is known to promote intestinal absorption of minerals, particularly calcium, as well as calcium mobilization. As used herein, “vitamin D” refers to vitamin D, cholecalciferol (vitamin D₃), ergocalciferol (vitamin D₂) and its biologically active metabolites and precursors such as, 1α,25-dihydroxyvitamin D; 25-OH vitamin D, its biological precursor; and 1α-hydroxyvitamin D, and analogues of the dihydroxy compound. The delivery system of the present invention may comprise one or more forms of vitamin D, for example, vitamin D₃ and vitamin D₂. Vitamin D can be obtained from various commercial suppliers, for example, cold water soluble Vitamin D₃ is available commercially from BASF Corporation (Paramus, N.J.) and Hoffmann-La Roche (Nutley, N.J.).

Other vitamins known in the art to help in the absorption and utilisation of calcium by the body include Vitamin C and Vitamin K, which can also be included in the delivery systems of the invention. “Vitamin C” as used herein, includes ascorbic acid (generally L-ascorbic acid) and sodium ascorbate. “Vitamin K” as used herein, refers to phylloquinone (vitamin K₁).

The delivery systems can also comprise minerals associated with calcium metabolism and bone health. Suitable magnesium salts include, but are not limited to, magnesium acetate, magnesium carbonate, magnesium carbonate hydroxide, magnesium chloride, magnesium citrate, magnesium glycerophosphate, magnesium hydroxide, magnesium lactate, magnesium oxide, magnesium phosphate (di- and triphosphate), magnesium sulphate, magnesium trisilicate, and combinations thereof. Magnesium glycerophosphate has the added benefit of also contributing a phosphorous component to the composition.

Phosphorous can contribute to bone density and can be included in the delivery system as phosphate salts of sodium, potassium, magnesium, iron, calcium, lithium or zinc, or as magnesium glycerophosphate.

Suitable copper sources include cupric sulphate, cupric carbonate, copper gluconate, cupric oxide, or a combination thereof. Manganese can be included as manganese gluconate, manganese sulphate, manganese amino acid chelates, or a combination thereof. Zinc can be included as zinc carbonate, zinc chloride, zinc citrate, zinc gluconate, zinc oxide, or a combination thereof. Boron can be included as boron alpha-ketoglutarate, boron aspartate, boron citrate, boron glycinate, boron picolinate, or a combination thereof.

The delivery systems may also comprise selenium and/or fluoride.

Isoflavones have also been reported to contribute to bone health and can be included in the delivery systems of the invention. Suitable isoflavones include, but are not limited to, naturally occurring soy isoflavones such as daidzein (4′,7-dihydroxyisoflavone), genistein (4′,5,7-trihydroxyisoflavone), and glycitein, which occur in a variety of forms (for example, in glycosidic and acetylated forms). Soy isoflavones are commercially available, for example, from Archer Daniels Midland (Decatur, Ill.). Synthetically derived isoflavones, such as ipriflavone (a synthetic 7-isopropoxyisoflavone) can also be used.

If desired, micronised or pre-coated forms of the above-described functional ingredients may be used.

Typically, the total amount of calcium and other functional ingredients constitute up to about 40% by weight of a delivery system. Thus, the amount of other functional ingredient(s) included in the delivery system will be dependent on the amount of calcium that is to be incorporated. In all cases, however, the amount of the one or more antacids included in the delivery system is greater on a weight % basis than the amount of any other single functional ingredient. In one embodiment of the present invention, the delivery systems incorporate between about 0.01% and about 20% by weight of other functional ingredient(s) in addition to calcium. In another embodiment, the delivery systems incorporate between about 0.01% and about 15% by weight of other functional ingredient(s) in addition to calcium. In another embodiment, the delivery systems incorporate between about 0.01% and about 10% by weight of other functional ingredient(s). In a further embodiment, the delivery systems incorporate between about 0.01% and about 5% by weight of other functional ingredient(s).

### 4. Bioavailability Enhancers

The present invention also contemplates the inclusion of bioavailability enhancers in the delivery systems. Such compounds are known in the art and act to increase the absorption of functional ingredients by the body. Bioavailability enhancers can be natural or synthetic compounds. In accordance with the present invention, the bioavailability enhancer is a natural compound.

Natural bioavailability enhancers include ginger, caraway extracts, pepper extracts and chitosan. The active compounds in ginger include 6-gingerol and 6-shogoal. Caraway oil can also be used as a bioavailability enhancer (U.S. Patent Application 2003/022838). Piperine is a compound derived from pepper (Piper nigrum or Piper longum) that acts as a bioavailability enhancer (see U.S. Pat. No. 5,744,161). Piperine is available commercially under the brand name Bioperine® (Sabinsa Corp., Piscataway, N.J.).

One or more of the above-described bioavailability enhancers may be included in the delivery systems in order to enhance the bioavailability of calcium and/or other functional ingredients. Typically, one or more bioavailability enhancer can be included in the delivery system in an amount between about 0.02% to about 0.6% by weight.

### Process for Preparing the Delivery System

In accordance with the present invention, the delivery systems remain flowable at temperatures below 100° C., which allows for full dispersion and incorporation of the calcium and the other optional functional ingredients into the matrix while minimising or preventing degradation of these compounds. Thus, although the actual methodology used to prepare the delivery systems may vary depending on the individual components selected to make up the matrix, the process of preparing the matrix comprises the step of incorporating the calcium and other optional functional ingredient(s) into the matrix at temperatures below 100° C. In one embodiment of the present invention, the process of preparing the matrix comprises the step of incorporating the functional ingredient(s) into the matrix at temperatures below about 75° C. In another embodiment, the process of preparing the matrix comprises the step of incorporating the functional ingredient(s) into the matrix at temperatures below about 65° C. In another embodiment, at least one functional ingredient is dispersed in the solvent component prior to admixture with the other matrix components.

Various standard methods known in the confectionery manufacturing industry can be used to prepare the delivery systems and selection of the appropriate method is considered to be within the ordinary skills of a worker in the art. Batch processes, such as kettle cooking, as well as continuous processes, such as direct steam injection jet cookers and indirect steam tubular heat exchangers, are suitable for preparing the delivery system.

The following description represents a general method of preparing a delivery system of the present invention.

Briefly, the process comprises the following steps: a blend of the hydrocolloid component and the sugar component, and optionally water, is prepared. A ratio of components is selected that will result in a final product with the desired moisture content (i.e. 10%-40%). The hydrocolloid(s) may be pre-hydrated in water or may be hydrated during this blending step. The blend is heated to a temperature of less than 100° C., for example between 60° C. and 80° C., such that all ingredients are incorporated. Alternatively, the sugar component, and optionally water, can be heated to a temperature of less than 100° C. (for example between 60° C. and 80° C.) prior to addition of the dry or pre-hydrated hydrocolloid(s) under shear. The temperature of the mixture is then reduced to between 50° C. and 80° C. The calcium source(s) and/or other optional functional ingredient(s) are dispersed or dissolved in solvent at or below 70° C., for example below 50° C. If required, one or more sources of mono- or divalent cations and one or more pH adjusting agents can be added to either, or both, of the above preparations. The two preparations are then combined. Flavourings and colourings may optionally be added after this step.

As an alternative to adding pH adjusting agents as indicated above, the pH of the matrix can be adjusted, as necessary, after combining the two preparations. Suitable methods of adjusting the pH of food products are known in the art and include, for example, the addition of buffers, acids or bases, such as citric acid, sodium citrate, phosphates, sodium hydroxide, potassium hydroxide or a combination thereof.

As indicated above, the final product has a moisture level between 10% and 40%, for example between 15% and 20%, and a water activity of less than 0.9.

In one embodiment of the invention, the process includes the step of heating the blend of hydrocolloid(s) and the sugar component (and optionally water) to a temperature between about 60° C. and about 70° C. In another embodiment, the process includes the step of heating the sugar component, and optionally water, to a temperature between about 60° C. and about 70° C. prior to addition, under shear, of the dry or pre-hydrated hydrocolloid(s). In a further embodiment, the process includes the step of dispersing or dissolving the calcium and/or other optional functional ingredient(s) in the solvent at a temperature between about 40° C. and about 50° C.

Once the matrix has been prepared as described above, it can then be moulded, for example, using the standard Mogul process or by injection-filling of preformed moulds. One skilled in the art will appreciate that the matrix can also be readily adapted to extrusion methods.

In final form, the delivery systems of the present invention are semi-solid, intermediate moisture systems, having some properties clearly identified with those of jellies and some properties that are similar to the jujube variety of confectioneries. The matrix of the delivery systems is thus formulated to be semi-solid at normal room temperature (i.e. at temperatures between about 20° C. and about 30° C.). It will be readily apparent that depending on the particular components selected for use in the preparation of the matrix, the amount of each to be included in the matrix may need to be manipulated within the ranges indicated in order to achieve a semi-solid, intermediate moisture product. One skilled in the art of confectionery design can readily determine which component(s) will need to be adjusted in order to achieve an end-product with these physical properties.

Similarly, it will be readily apparent to one skilled in the art that variations can be made to the described process dependent on the type and the actual amount of each component used (within the given ranges) in order to obtain an end product with the described properties. For example, if the hydrocolloid comprises a starch, it is known in the art that the gelatinisation temperature of the starch may be affected when certain sugars and sugar alcohols are used. If required, therefore, the starch and the sugar component can be heated above 100° C. to allow full gelatinisation of the starch to occur and the desired moisture content to be reached. The temperature of the mixture can then be reduced to between 50° C. and 80° C. prior to addition of the functional ingredient(s) and optionally flavourings and colourings.

As is known in the art, modified celluloses, such as methylcellulose and hydroxypropyl methylcellulose, have unique properties resulting in the ability to delay hydration of these carbohydrates during preparation processes. Thus, when these compounds are used a “delayed hydration technique” may be employed in which the modified cellulose is first dispersed in the solvent component of the matrix and then mixed with the other components in aqueous solution. The hydration of the modified cellulose then takes place gradually as the processing is complete and the moulded matrix cools. Delayed hydration and non-aqueous fluid carrier techniques using modified celluloses are standard in the art.

Similarly, the choice of hydrocolloid can affect the set up temperature of the matrix. The use of a combination of starch, gelatine and gellan, for example, can provide a matrix set-up temperature of about 35° C., as can a combination of starch, gelatine and pectin. In contrast, the use of other hydrocolloids or combinations of other hydrocolloids with or without gelatine or gellan, may alter the set up temperature of the matrix. For example, the use of starch in combination with locust bean gum or carageenan often results in set up temperatures of around 60° C. The choice of hydrocolloid is thus dependent on the functional ingredient(s) to be incorporated into the matrix. Temperature sensitive functional ingredients will require a hydrocolloid or hydrocolloid mixture that provides a low set up temperature (such as the gelatine:gellan or gelatine:pectin mixtures described above), whereas other hydrocolloids or mixtures thereof can be used with functional ingredients that can tolerate higher temperatures.

The manner in which the individual components are combined may also be varied although typically at least one of the functional ingredients is dispersed in solvent prior to addition to the remainder of the components. For example, the sugar component may be heated with the water and salts prior to addition of the hydrocolloid(s). Similarly, when two or more hydrocolloids are being used, they do not have to be added to the mixture at the same time. One hydrocolloid and part of the sugar component could be mixed and heated prior to being blended with the other hydrocolloid and remainder of the sugar component. Alternatively, one hydrocolloid and the sugar component could be mixed and heated prior to addition of the second hydrated hydrocolloid, or one hydrocolloid may be added to the solvent component and then blended with the second hydrocolloid and sugar component. These and other variations are considered to be within the scope of the present invention.

### Testing the Delivery System

### 1. Physical Properties

One skilled in the art will appreciate that molecular interaction between one or more of the functional ingredient and the matrix may affect the physical attributes of the final product. As is standard in the art, therefore, a sample of the delivery system incorporating the calcium source and optionally other functional ingredient(s) can be prepared prior to large-scale production and tested in order to determine whether the matrix retains the desired physical properties, i.e. substantially uniform dispersion of the calcium and other functional ingredients, less than 20% degradation of these compounds during the preparation of the matrix and water activity of less than 0.9.

For example, dispersion of the calcium in the final delivery system can be determined by dividing a single unit of the delivery system into several subunits and analysing the content of calcium in each subunit, for example as a % by weight. The levels of calcium can readily be measured by standard analytical techniques such as mass spectrometry, UV or IR spectrometry, or chromatographic techniques, such as gas chromatography or high-performance liquid chromatography (HPLC). If the % by weight of calcium in each subunit is similar, then the calcium is said to be substantially uniformly dispersed throughout the product. One skilled in the art will appreciate that the % by weight need not be identical for each subunit to indicate substantially uniform dispersion. In accordance with the present invention, the % by weight of calcium for each subunit of the final delivery system varies by less than 2%. In one embodiment, the % by weight of calcium for each subunit of the final delivery system varies by less than 1.5%. In other embodiments, the % by weight of calcium for each subunit varies by less than 1% and by less than 0.5%.

The dispersion of other functional ingredients incorporated into the delivery system can also be measured as described above.

Similarly, the degradation of the functional ingredient(s) can be determined by standard analytical techniques taking into account the total amount of each compound included in the preparation of the matrix. Many functional ingredients degrade to yield specific breakdown products, the presence or absence of which can be determined in the final product using standard techniques, such as spectrophotometric and chromatographic techniques, e.g. gas chromatography and HPLC. As indicated above, the degradation of the functional ingredients is minimised during the preparation of the delivery system and is less than about 20% in the final product.

The water activity (a_{w}) of the final product can also be analysed by standard techniques. The a_{w} of a food product is a physical property that has direct implications on the microbial safety of the product and influences storage stability. Lower a_{w} values generally indicate a food product that is more stable and more resistant to microbial contamination than one with a high a_{w} value due to the requirement for water of most microbes and the fact that most deteriorative processes in food products are mediated by water. As is known in the art, the a_{w} value of a_{w} food product is the ratio of the water vapour pressure of the product (p) to that of pure water (pₒ) at the same temperature, i.e. a_{w}=p/pₒ. In accordance with the present invention, the water activity of the final delivery system is less than about 0.9, for example between about 0.5 and about 0.7.

Other parameters, such as the release rate of the functional ingredients from a delivery system can also be tested by standard methods (for example, the USP Basket Method or Paddle Method; see U.S. Pharmacopoeia XXII (1990)). Typically, a sample of the delivery system containing a known amount of functional ingredient(s) (for example, a unit dose) is placed in an aqueous solution of a predetermined pH, for example around pH 1.2 to simulate stomach conditions and/or around pH 7.4 to simulate colon conditions. The suspension may or may not be stirred. Samples of the aqueous solution are removed at predetermined time intervals and are assayed for their content of the calcium and other optional functional ingredients by standard analytical techniques, such as those indicated above.

In addition, the delivery system may undergo testing to evaluate such factors as the microbial content of the product and the shelf-life of the product. Such quality control testing is standard in the art and can be conducted using known methods.

For example, microbial analysis of the delivery system can be conducted using techniques approved by the appropriate regulatory board, such as those described in “The Compendium of Analytical Methods: HPB Methods for the Microbiological Analysis of Foods” issued by the Health Products and Food Branch of Health Canada. Shelf life is typically evaluated using accelerated shelf life tests in which the stability of the system and the degradation of the functional ingredients contained therein is analysed under conditions that are known to accelerate the degradation of food products and can be correlated to the stability of the product under normal storage conditions.

Texture measurements can also be made to determine whether the delivery system has the required gel strength/hardness. Gel strength or hardness can be measured either directly (expressed as grams force) and indirectly (expressed as a viscosity), or both.

Methods of measuring gel hardness are known in the art. For example, a Kramer single blade shear cell can be used. In this test, a shear blade is driven down at a constant speed through a sample of the delivery system and the peak force as the blade cuts through the sample is measured. The test force is typically reported in kilograms-force. Various machines are available to conduct such testing, for example, a Universal Testing machine such as that available from Instron or Stable Micro Systems (e.g. the Model TA.HD Texture Analyzer).

Gel hardness can also be measured using a standard Brookfield viscometer (e.g. the Model RVDV), which measures the force required to cut through a gelled liquid. A spindle rotating at a set speed is slowly lowered into a sample of the delivery system and the torque required for the spindle to “cut” through the sample is measured. Temperature is important to obtain an accurate viscosity reading and thus the samples are usually tempered to 21° C. to 24° C. prior to testing. The cutting force or torque reading on the viscometer is an empirical measure of gel strength and is reported in centipoise (cps).

Another method useful for measuring sensory texture utilises the Hamann Torsion/Vane Gelometer. This system provides fracture shear stress and shear strain values and real time test graphs of stress vs. strain or angular deformation. Stress (strength) and strain (deformability) are not “geometrically coupled” as in most traditional (empirical) textural tests, therefore, the strain measurement remains unaffected by the magnitude of the stress measurement. Strain has been found to be the best indicator of gelling quality for proteins and hydrocolloids, as this parameter is less sensitive to concentration effects, and is also a good indicator of the perceived “rubberiness” of food gels. Strain values also predict machining characteristics of food gels, such as ease of slicing. Furthermore, the sample shape does not change during testing with the Torsion Gelometer, thus minimal fluids will be forced from the sample during testing and the gel itself is tested rather than a dehydrated derivative. The mode of failure in torsion testing yields important information about the texture of the sample. Test samples of the delivery system are formed in either cylindrical molds (tubes) for subsequent milling, which eliminates surface skin effects, or in a dumbbell mold. Samples are then cut to a standard length (for example, 1 inch) and loaded into the measuring cell for testing. Data collection continues for a time past the breaking of the sample (peak stress or Fracture Point). Stress (in kPa), strain, rigidity modulus (G=stress/strain) and slope ratio at failure can be measured in this method

Palatability can also be tested using standard techniques. Methods of evaluating the organoleptic properties of foods are well-known in the art. For example, sensory evaluations can be performed using individuals who are spatially separated from each other, for example, in individual partitioned booths, as testers and a hedonic nine-point scale that ranges from 1 (most disliked) to 9 (most liked), with 5 indicating no preference [Larmond, Laboratory methods for Sensory Evaluation of Foods, Research Branch of Agriculture Canada (1977)]. Odour and taste are generally evaluated under a red light, which masks any differences in the colour of the product. Another nine-point hedonic scale test can be carried out under normal light to evaluate the acceptability of the appearance of the product.

### 2. Efficacy

The delivery systems of the present invention can be tested for efficacy in vivo. Typically, the efficacy is tested by conducting bioavailability studies using standard techniques in the pharmaceutical art, such as peak plasma levels and pharmokinetic analyses (see, for example, Enna, et al., Current Protocols in Pharmacology, J. Wiley & Sons, New York, N.Y.).

Bioavailability studies are usually conducted by administering to groups of subjects various doses of the delivery system under study over a pre-determined period of time and comparing plasma levels of calcium in these groups at varying intervals with an appropriate control or controls. Appropriate controls include groups of subjects taking recommended doses of competitor's products (positive control) and/or groups of subjects taking a placebo or no supplement (negative control). The subjects may or may not have fasted prior to administration of the doses of the delivery system. Single dose or multiple dose studies may be conducted. The studies can also be used to monitor any side-effects of the dosing regimens of the delivery system under investigation by compiling reports of any adverse effects encountered during the course of the study and comparing them to side-effects reported by the control group(s). Optimal dosing schedules can also be determined in this manner.

Studies to determine that the combination of functional ingredients in a delivery system bring about the desired effect, for example increased bone density, in a subject can also be conducted in a similar manner to the bioavailability studies indicated above. Such studies are routine in the art and can be readily designed and conducted by a skilled technician.

### Format of the Delivery System

The present invention contemplates various formats for the delivery systems. For example, the delivery systems may be in the form of a confectionery, such as a jujube, in which case it may be formulated alone or it may further comprise a coating, such as a chocolate or yoghurt coating. Preparation of jujube or jelly type confectionery products are known in the art and include, for example, the use of moulds, injection-filling of pre-formed packages and extrusion processes. It will be readily apparent to one skilled in the art that such standard techniques can be applied to prepare a wide variety of different shaped confectioneries.

The present invention further contemplates the delivery system as a filling or a coating, for example, for baked goods such as wafers or cookies. For example, the matrix can be used as a layer between two wafers, or a jelly layer on the top of a cookie or sponge, in which case the product may be further coated with a chocolate or other flavoured coating, if desired, as described above for confectionery products. Alternatively, the matrix may be used to fill doughnut type baked goods. Methods of filling and coating baked goods are also well known in the art.

### Administration and Use

Calcium and the other optional functional ingredients are incorporated into the delivery system at levels sufficient to affect the structure or function of the body when taken regularly. Such levels are generally based on the Recommended Daily Amount (RDA). RDAs for calcium and representative examples of other functional ingredients contemplated by the present invention are provided in Table 2.

TABLE 2
Recommended Daily Amount (RDA) for Calcium and other Functional
Ingredients

| Functional | RDA | | | | | |
|---|---|---|---|---|---|---|
| Ingredient | Men | | Women | | Children* | |
| Calcium | 1000-1200 | mg | 800-1200 | mg | 500-1300 | mg |
| Boron | Not Determinable | | Not Determinable | | Not Determinable | |
| Copper | 1.5-3.0 | mg | 1.5-3.0 | mg | 0.7-2.5 | mg |
| Magnesium | 280-350 | mg | 280-355 | mg | 80-400 | mg |
| Manganese | 2.3 | mg | 1.8-2.6 | mg | 1.2-2.2 | mg |
| Phosphorus | 700 | mg | 700-1200 | mg | 460-1250 | mg |
| Zinc | 15 | mg | 12-19 | mg | 10-15 | mg |
| Vitamin C | 60 | mg | 50-95 | mg | 40-60 | mg |
| Vitamin D | 5-10 | μg | 5-10 | μg | 10 | μg |
| Vitamin K | 70-80 | | 60-65 | μg | 15-65 | |

*Male and female < 18 y

The delivery systems of the invention can be formulated in various unit sizes depending on the amount of calcium (and other optional functional ingredients) to be incorporated therein and on the requirements of the target consumer. For example, smaller units may be required for children and animals than for adult humans. The delivery systems of the present invention can be formulated to have a unit size between about 3 grams and about 30 grams. In one embodiment, a unit of the delivery system is between about 3 and about 20 g. In another embodiment, a unit of the delivery system is between about 3 and about 15 g. In another embodiment, a unit of the delivery system is between about 3 and about 10 g. Where appropriate, the delivery systems can be provided in a multi-dose format that is pre-scored into unit doses.

It is understood that the total daily intake of calcium may be based on administration of one unit of the delivery system, or it may be based on administration of more than one unit. The amount of calcium in a single unit will thus vary depending on the size of the units and the number to be administered daily. For example, one unit of the delivery system may contain a fraction of the RDA of calcium or it may provide the entire RDA of calcium. Similarly, one unit of the delivery system may contain a fraction of the RDA of any other functional ingredients, where applicable, or the entire RDA for these components.

The delivery systems of the present invention can be used as a dietary calcium supplement. The present invention thus provides for a method of supplementing calcium intake in a mammal by administering an effective amount of the calcium delivery system. Calcium supplementation can promote bone health, for example by helping to maintain bone strength, increase bone mass/density and/or growth and prevent bone loss. Calcium supplementation has also been shown to reduce mild hypertension, as well as reducing the risk of hypertension in pregnant women (Griffith L. E., et al., American J Hypertens., 1999, 12: 84-92), and has shown efficacy in decreasing moderate to severe cyclically recurring premenstrual symptoms. The delivery systems of the present invention, therefore, are suitable for use in subjects suffering from high blood pressure and in women suffering from premenstrual syndrome.

Examples of suitable combinations of functional ingredients for delivery systems intended to promote bone health, based on a daily intake of two units of the delivery system include, but are not limited to, one or more source of calcium that provides 250-500 mg of calcium (on an elemental basis) per unit in combination with one or more of:

2.5-5 μg Vitamin D per unit
20-40 μg Vitamin K per unit
40-250 mg Vitamin C per unit
225-450 μg copper* per unit
0.75-2.0 mg fluoride per unit
75-200 mg magnesium* per unit
0.45-1.3 mg manganese* per unit
200-600 mg phosphorus* per unit
10-30 μg selenium* per unit
2.75-6.5 mg zinc* per unit
(* on an elemental basis)

Another example would be delivery systems comprising one or more source of calcium that provides 250-500 mg of calcium (on an elemental basis) per unit in combination with one or more of:

2.5-5 μg Vitamin D per unit
20-40 μg Vitamin K per unit
225-450 μg copper* per unit
0.75-2.0 mg fluoride per unit
0.45-1.3 mg manganese* per unit
2.75-6.5 mg zinc* per unit
(* on an elemental basis)

A further example would be delivery systems comprising one or more source of calcium that provides 250-500 mg of calcium (on an elemental basis) per unit in combination with one or more of:

2.5-5 μg Vitamin D per unit
20-40 μg Vitamin K per unit
40-250 mg Vitamin C per unit
75-200 mg magnesium* per unit
0.45-1.3 mg manganese* per unit
200-600 mg phosphorus* per unit
10-30 μg selenium* per unit
(* on an elemental basis)

The above delivery systems can also include the trace element boron and/or inulin and/or other fructooligosaccharides. Delivery systems that are formulated for women can also optionally include 12.5-325 mg isoflavones (such as ipriflavone and/or soy isoflavones, singly or in combination) per unit.

Certain groups of the general population can obtain particular benefit from the calcium delivery systems of the present invention. For example, calcium supplementation to help increase bone mass is particularly important in children and teenagers, as well as osteoporosis sufferers. Similarly, calcium supplementation to help prevent bone loss or development of osteoporosis is important in adults, especially in pre- and post-menopausal women and elderly men.

In addition, maintenance of bone health is of prime importance to athletes and other individuals who regularly partake in exercise. Optimal nutrition, including adequate intake of minerals such as calcium, is also of high importance to those individuals who are trying to attain peak performance in their sport. One embodiment of the present invention thus contemplates the use of the calcium delivery systems for sports nutrition purposes. Sports nutrition is associated with the intake of functional ingredients that affect various factors relating to an individual's endurance, performance, recovery, energy levels, weight maintenance, and the like. The calcium delivery system of the invention can be utilised in sports nutrition to enhance bone health and thereby decrease the risk of stress fractures and osteoporosis, which are common amongst elite athletes, in particular female athletes. The calcium delivery systems can also help to prevent or ameliorate other conditions may affect an individual's ability to perform or recover, such as hypertension and premenstrual syndrome.

Endurance, performance and recovery can be also improved through the administration of a sports nutrition delivery system that comprises one or more functional ingredients that help muscle performance, muscle endurance, increase fine motor control, and/or decrease muscle tremor. As calcium is known to play a role in muscle contractility and nerve transmission, the calcium delivery system of the invention may be used by athletes to help improve endurance, performance and/or recovery.

Regulation of metabolism can be useful in sports nutrition, for example to promote endurance and/or performance and recovery, or to increase energy and/or modulate weight loss. Calcium is known to play a role in fat storage (see, for example, Zemel, M. B., J Am Coll Nutr., 2002, 21:146S-151S) and high calcium diets can lead to increased fat metabolism. Thus, the present invention further contemplates the use of the calcium delivery system to help mobilise body fat stores and/or modulate fat metabolism.

In view of the importance of calcium supplementation to the general population, it is further contemplated that “dual-purpose” calcium delivery systems can be designed that comprise a combination of functional ingredients, the main component of which is calcium. The remaining functional ingredients of the combination being selected to produce a specific physiological effect. Non-limiting examples of such effects include the improvement of an individual's endurance, performance, recovery, energy levels, weight maintenance, and the like, as described above, as well as those effects outlined in Table 1.

The delivery systems can be formulated for administration to humans or other animals. For administration to humans, flavours and formats that appeal to the particular group of consumers being targeted can be employed. For example, delivery systems that are formulated with confectionery-like qualities and flavours are appealing to children who are often resistant to taking medications or supplements due to unpleasant tastes or mouthfeel.

Similarly, the delivery systems can be formulated for administration to a non-human animal using flavours that more typically appeal to non-human animals, for example, fish, poultry or meat flavours. Administration of functional ingredients to an animal in conventional solid dosage forms, such as tablets and capsules, can be problematic in that the animal often expels them, and multiple dosing is often difficult because the animal learns to resist the dosing procedure. It will be readily apparent that the delivery system of the present invention, which is formulated as a foodstuff, is ideally suited for administration of calcium to animals.

### Kits

The present invention additionally provides for kits containing a delivery system for administration to a human or non-human animal. The kit would provide an appropriate dosing regimen over a prescribed period for the calcium and other functional ingredient(s) contained in the delivery system.

The kits of the invention comprise one or more packages containing the delivery system and may further comprise a set of instructions, generally written instructions, relating to the use and dosage of the calcium and other optional functional ingredient(s) contained in the delivery system. The instructions typically include information as to the appropriate dosage and dosing schedule for the functional ingredients in terms of units of the delivery system. The packages containing the delivery system may in the form of unit doses, bulk packages (for example, multi-dose packages) or sub-unit doses. The doses may be packaged in a format such that each dose is associated, for example, with a day of the week. There may also be associated with the kit a notice in the form prescribed by a governmental agency regulating the manufacture, use or sale of biological products, which notice reflects approval by the agency of manufacture, use or sale for human or animal administration.

To gain a better understanding of the invention described herein, the following examples are set forth. It should be understood that these examples are for illustrative purposes only. Therefore, they should not limit the scope of this invention in any way. All percentages throughout the specification and claims are by weight of the final delivery system unless otherwise indicated.

### EXAMPLES

### Example 1

### Calcium Delivery Systems

The delivery systems described below are formulated to have a final pH between 5.0 and 9.0, more typically between 6.5 and 8.5. The delivery systems have a final a_{w} between about 0.5 and about 0.6.

1.1 Delivery System for Calcium #1

| Ingredient | % by Weight |
|---|---|
| Glycerol | 39.04% |
| Propylene Glycol | 2.32% |
| Calcium Carbonate | 15.00% |
| 63 DE Corn syrup | 10.74% |
| High Fructose Corn Syrup | 12.61% |
| Gelatine | 7.07% |
| Pectin | 0.27% |
| Sweetening agents | 0.05% |
| Modified starch | 2.21% |
| Flavour | 0.12% |
| Colour | 0.29% |
| Water | 10.28% |
| Total: | 100.00% |

The final moisture content of final delivery system was approximately 16% by weight.

1.2 Delivery System for Calcium #2

| Ingredient | % by Weight |
|---|---|
| Glycerol | 46.17% |
| Propylene Glycol | 1.66% |
| Calcium Carbonate | 12.04% |
| 63 DE Corn syrup | 9.15% |
| High Fructose Corn Syrup | 10.74% |
| Gelatine | 5.53% |
| Pectin | 0.26% |
| Sweetening agents | 0.04% |
| Modified starch | 1.77% |
| Flavour | 0.15% |
| Colour | 0.30% |
| Water | 12.20% |
| Total: | 100.01% |

### 1.3 MCHC* Delivery System

| Ingredient | % by Weight |
|---|---|
| Glycerol | 21.90% |
| Propylene Glycol | 1.56% |
| MCHC* | 11.98% |
| 63 DE Corn syrup | 21.90% |
| High Fructose Corn Syrup | 28.15% |
| Citric acid | 0.62% |
| Gelatine | 6.26% |
| Gellan | 0.25% |
| Sweetening agents | 0.05% |
| Fructo-oligosaccharides | 3.13% |
| Flavour | 0.50% |
| Colour | 0.27% |
| Water | 3.44% |
| Total: | 100.01% |

*MCHC = Microcrystalline hydroxyapatite complex; a mixed mineral calcium source derived from bone.

### 1.4 Delivery System for Calcium and Vitamin D #1

The following delivery system was formulated to deliver 1.2 g calcium carbonate and 10 μg vitamin D in a 10 g product.

| Ingredient | % by Weight |
|---|---|
| Glycerol | 46.17% |
| Propylene Glycol | 1.66% |
| Vitamin D | 0.04% |
| Calcium Carbonate | 12.00% |
| 63 DE Corn syrup | 9.15% |
| High Fructose Corn Syrup | 10.74% |
| Gelatine | 5.53% |
| Pectin | 0.26% |
| Sweetening agents | 0.04% |
| Modified starch | 1.77% |
| Flavour | 0.15% |
| Colour | 0.30% |
| Water | 12.20% |
| Total: | 100.01% |

### 1.5 Delivery System for Calcium, Vitamin D and Isoflavones

The following delivery system was formulated to deliver 1.2 g calcium carbonate, long vitamin D and 50 mg isoflavone in a 10 g product.

| Ingredient | % by Weight |
|---|---|
| Glycerol | 44.90% |
| Propylene Glycol | 1.67% |
| Vitamin D | 0.04% |
| Calcium Carbonate | 12.00% |
| Soy isoflavone preparation | 1.25% |
| 63 DE Corn syrup | 9.15% |
| High Fructose Corn Syrup | 10.74% |
| Gelatine | 5.53% |
| Pectin | 0.26% |
| Sweetening agents | 0.04% |
| Modified starch | 1.77% |
| Flavour | 0.15% |
| Colour | 0.30% |
| Water | 12.20% |
| Total: | 100.01% |

### 1.6 Delivery System for Calcium and Vitamin D #2

The following delivery system was formulated to deliver about 1.75 g calcium lactate and 3.5 μg Vitamin D in a 5.5 g product.

| Ingredient | % by Weight |
|---|---|
| Glycerol | 31.6658% |
| Propylene Glycol | 0.9223% |
| Vitamin D (10 0000 IU/g) | 0.0252% |
| Calcium lactate | 35.0476% |
| High Fructose Corn Syrup | 13.8346% |
| Gelatine | 3.8429% |
| Pectin | 0.1783% |
| Sweetening agents | 0.0307% |
| Modified starch | 1.2297% |
| Flavour | 0.1045% |
| Colour | 0.2060% |
| Water | 12.9123% |
| Total: | 100.0000% |

The above calcium formulations were prepared by the following general method:

The glycerol and propylene glycol were blended and the calcium source dispersed therein and the blend warmed to 40-50° C. The sugar syrups were blended with the water and warmed to 60-70° C. The gelatine, pectin, sweetening agents and other dry ingredients were preblended and introduced into the syrup(s) under shear. The calcium blend was then uniformly blended with the gelatine preparation. Flavour and colour were then added and the whole maintained between 40° C. and 55° C. When additional functional ingredients are included in the delivery system, they are either added into the glycerol and propylene glycol together with the calcium source, or the more stable functional ingredients can be combined with the other dry ingredients prior to addition to the syrups.

### 1.7 Sports Nutrition Calcium Delivery System

The following delivery system was formulated to deliver between 500 and 600 mg of calcium carbonate.

| Ingredient | % by Weight |
|---|---|
| Glycerol | 33.0-43.0% |
| High fructose corn syrup | 13.0-19.0% |
| 63 DE corn syrup | 11.0-16.0% |
| Water | 8.0-12.0% |
| Gelatine | 5.0-7.0% |
| Calcium carbonate | 3.5-6.5% |
| Blend of carnitine, ginseng, green | 3.0-5.0% |
| tea, taurine, tyrosine and yerbamate | |
| Propylene Glycol | 2.0-3.0% |
| Modified starch | 1.5-3.0% |
| Caffeine | 1.0-2.0% |
| Methylcellulose | 0.8-2.0% |
| Flavour | 0.5-3.0% |
| Colour | 0.01-1.0% |
| Pectin | 0.01-0.3% |
| Artificial sweetener | 0.01-0.2% |
| Vitamin D | 0.005-0.1% |
| Citric acid | 0.0-0.5% |

The above formulation was prepared by the following process. The glycerol and propylene glycol were blended. The calcium, methylcellulose and proprietary blend of actives are preblended together then incorporated into the glycerol/propylene glycol and the blend warmed to 40-50° C. When the vitamin D is used in powder form it can be added to the preblend, when used in liquid form, it can be added to the glycerol /propylene glycol prior to adding the dry preblend. The caffeine was dissolved in water heated to between 65° C. and 85° C. The sugar syrups were then incorporated and the temperature adjusted to 60-70° C. The gelatine, pectin, starch and sweetening agents were preblended and introduced into the syrup(s) under shear. The calcium blend was then uniformly blended with the gelatine preparation. Flavour and colour were then added and the whole maintained between 40° C. and 55° C.

### Example 2

### Delivery Systems Using Other Functional Ingredients

The following delivery systems (formulated using functional ingredients other than calcium) demonstrate how the components of the matrix can be varied. These systems can be readily adapted for calcium delivery by a worker skilled in the art, by replacing the listed functional ingredients with a source of calcium and optionally, one or more other functional ingredient, in accordance with the present invention. A worker skilled in the art will recognize that the use of pH modifying or buffering ingredients included when formulating with specific functional ingredients may not be required when adapting the formulations to deliver calcium. Similarly, a worker skilled in the art will recognise that the ingredients in the following formulations may need to be adjusted when including large amounts of cationic calcium in the system. The moisture content of the following delivery systems was between about 13% and about 17% by weight.

### 2.1 Ingredient % by Weight

| Ingredient | % by Weight |
|---|---|
| Glycerol | 14.57% |
| Propylene Glycol | 5.30% |
| Functional ingredients* | 13.38% |
| Corn Syrup 62DE | 31.79% |
| Sucralose | 0.04% |
| Modified Starch | 2.65% |
| Potassium citrate | 2.15% |
| High fructose corn syrup | 9.27% |
| Water | 14.57% |
| Gelatine 100 bloom type B | 1.32% |
| Gelatine 250 bloom type A | 3.97% |
| Gellan (Kelcogel ® LT100) CP Kelco | 0.32% |
| Colour | 0.21% |
| Flavour | 0.45% |
| Total: | 100.00% |

*creatine monohydrate (11.71%) and dimethylglycine (1.67%)

### 2.2 Ingredient % by Weight

| Ingredient | % by Weight |
|---|---|
| Glycerol | 12.57% |
| Propylene Glycol | 4.19% |
| Functional ingredient (arginine) | 14.02% |
| Maltitol solution | 33.52% |
| Modified Starch | 2.79% |
| Potassium citrate | 1.17% |
| Sucralose | 0.04% |
| High fructose corn syrup | 9.78% |
| Water | 15.37% |
| Gelatine 250 bloom type A | 5.59% |
| Gellan (Kelcogel ® LT100) CP Kelco | 0.28% |
| Colour | 0.168% |
| Flavour | 0.503% |
| Total: | 100.00% |

### 2.3 Ingredient % by Weight

| Ingredient | % by Weight |
|---|---|
| Glycerol | 13.82% |
| Propylene Glycol | 5.53% |
| Functional ingredients* | 11.02% |
| Isomalt syrup | 33.17% |
| Sucralose | 0.055% |
| Modified Starch | 2.76% |
| Potassium citrate | 2.24% |
| High Fructose Corn syrup | 9.68% |
| Water | 15.20% |
| Gelatine 250 bloom type A | 5.53% |
| Gellan (Kelcogel ® LT100) CP | 0.33% |
| Colour | 0.08% |
| Flavour | 0.08% |
| Total: | 100.00% |

*creatine monohydrate (4.59%), conjugated linoleic acid (CLA; 4.59%), lecithin (1.05%), N,N, dimethylglycine (0.47%), rhodiola/seabuckthorn extract solution (0.21%) and chromium chelate (0.11%).

### 2.4 Ingredient % by Weight

| Ingredient | % by Weight |
|---|---|
| Glycerol | 14.82% |
| Propylene Glycol | 5.39% |
| Functional ingredient (creatine | 11.91% |
| monohydrate) | |
| Corn Syrup 62DE | 32.33% |
| Sucralose | 0.04% |
| Modified Starch | 2.70% |
| Potassium citrate | 2.19% |
| High fructose corn syrup | 9.43% |
| Water | 14.82% |
| Gelatine 100 bloom type B | 1.34% |
| Gelatine 250 bloom type A | 4.04% |
| Gellan (Kelcogel ® LT100) CP | 0.33% |
| Kelco | |
| Colour | 0.21% |
| Flavour | 0.46% |
| Total: | 100.00% |

The above formulations were prepared by the following general method:

Glycerol and propylene glycol were first blended and at least one functional ingredient was added. The blend was heated to 65-70° C. In a separate container, gelatine and gellan were blended together. The fructose syrup and water were mixed and heated to 60° C., after which the gelatine:gellan mixture was added with constant agitation. The mixture was then heated to 75° C. to allow the components to dissolve. In a third container, the syrup was warmed to 30-35° C. and the sucralose, potassium citrate, other functional ingredients and starch were then blended in. The syrup mixture was combined with the gelatine:gellan mixture and heated to 75-80° C. until the moisture content was reduced and the desired solids level achieved. The glycerol mixture was then added together with the colour and flavour additives. The delivery system was then moulded using standard techniques.

### 2.5 Ingredient % by Weight

| Ingredient | % by Weight |
|---|---|
| Glycerol | 27.9990% |
| Propylene Glycol | 3.4145% |
| Potassium Hydroxide | 0.1208% |
| Functional ingredient (creatine | 24.0154% |
| monohydrate) | |
| High Fructose Corn Syrup | 15.7068% |
| Corn syrup | 14.7962% |
| Modified Starch | 2.5040% |
| Water | 3.9836% |
| Potassium phosphate | 0.4234% |
| Sucralose | 0.0381% |
| Potassium citrate | 0.9526% |
| Gelatine Type A | 4.7803% |
| Pectin | 0.2732% |
| Flavour | 0.5464% |
| Colour | 0.2982% |
| Total: | 100.0000% |

The following method was used to prepare the above delivery system. Glycerol and propylene glycol were first blended and the creatine was added. The blend was heated to 45-50° C. In a separate container, the gelatine, pectin, starch and sucralose were blended together. The fructose and glucose syrups and water were mixed and heated to 60° C., after which the salts and pH modifying agents were added with constant agitation and heated to 60-70° C. to dissolve the solids. The powder blend was then incorporated into the syrup mixture using high shear. Finally, the creatine mixture was added, together with the colour and flavour additives, and blended. The delivery system was then moulded using standard techniques.

### 2.6 Ingredient % by Weight

| Ingredient | % by Weight |
|---|---|
| Glycerol | 16.67% |
| Propylene Glycol | 7.86% |
| Functional ingredients* | 9.36% |
| Maltitol syrup | 35.86% |
| High fructose corn syrup | 15.73% |
| Sucralose | 0.06% |
| Modified Starch | 3.15% |
| Potassium citrate | 1.42% |
| Potassium hydroxide | 0.92% |
| Water | 1.38% |
| Gelatine | 6.29% |
| Pectin | 0.31% |
| Colour | 0.3% |
| Flavour | 0.74% |
| Total: | 100.00% |

*Conjugated linoleic acid (Clarinol 80; 7.86%), citrus aurantium (0.5%), inulin (0.63%), caffeine (0.25%), mixed tocopherols (0.04%) and ascorbic acid (0.03%).

The following method was used to prepare the above delivery system. The glycerol and propylene glycol were first blended together. At least one functional ingredient was then added and the resultant mixture was warmed to 60-70° C. In another container, the syrups, water, potassium citrate and potassium hydroxide were combined and warmed to 60-70° C. The starch, gelatine, pectin, sucralose and remaining functional ingredients were pre-blended then added to the syrup mixture under high shear. This mixture was combined with the glycerol mixture and the temperature maintained at 60-70° C. until the moisture content was reduced sufficiently to give the desired solids level. Colour and flavour were added and the mixture was then moulded using standard techniques.

### 2.7 Ingredient % by Weight

| Ingredient | % by Weight |
|---|---|
| Glycerol | 15.97% |
| Propylene Glycol | 5.51% |
| Functional ingredient (creatine | 16.71% |
| monohydrate) | |
| 63 DE Corn syrup | 21.20% |
| High Fructose Corn Syrup | 24.78% |
| Gelatine 250 Bloom Type A | 5.51% |
| Gellan | 0.33% |
| Sucralose | 0.06% |
| potassium citrate | 1.40% |
| Modified Starch | 2.75% |
| Water | 4.96% |
| Flavour | 0.56% |
| Colour | 0.28% |
| Total: | 100.00% |

The following method was used to prepare the above delivery system. Creatine was added to a mixture of glycerol and propylene glycol, and heated to 40-60° C. The syrups were blended with water and the dry ingredients were mixed into the syrup mixture. The combined mixture was then heated to at least 80° C. Alternatively, the blended dry ingredients can be blended in with simultaneous live steam injection to reach at least 80° C. The solid content was then adjusted by addition of water if necessary to provide a final moisture content of between about 10% to about 30%. At this point, the temperature of the syrup mixture was lowered to between 50° C. and 80° C. and the glycerol-glycol mixture was added. Colour and/or flavouring additives were then added and the delivery system was injection filled into the preformed packaging.

### 2.8 Ingredient % by Weight

| Ingredient | % by Weight |
|---|---|
| Glycerol | 27.96% |
| Propylene glycol | 3.44% |
| Potassium hydroxide (45%) | 0.30% |
| Functional ingredient (creatine | 24.07% |
| monohydrate) | |
| Corn syrup 63DE | 13.34% |
| High fructose corn syrup | 15.65% |
| Water | 6.30% |
| Potassium phosphate | 0.43% |
| Potassium citrate | 0.96% |
| Sucralose | 0.03% |
| Gelatine | 7.11% |
| Flavour | 0.14% |
| Colour | 0.27% |
| Total: | 100.00% |

### 2.9 Ingredient % by Weight

| Ingredient | % by Weight |
|---|---|
| Glycerol | 26.32% |
| Propylene glycol | 3.43% |
| Potassium hydroxide (45%) | 0.23% |
| Functional ingredient (creatine | 24.03% |
| monohydrate) | |
| Corn syrup 63DE | 14.24% |
| High fructose corn syrup | 16.72% |
| Water | 4.04% |
| Potassium phosphate | 0.43% |
| Potassium citrate | 0.96% |
| Sucralose | 0.04% |
| Gelatine | 9.15% |
| Flavour | 0.14% |
| Colour | 0.27% |
| Total: | 100.00% |

The delivery systems of Examples 2.8 and 2.9 were prepared as follows. Glycerol and propylene glycol were first blended and the creatine was added. The blend was heated to 45-50° C. The syrups, water, salts and pH modifying agents were mixed and heated to 60-70° C. with constant agitation to dissolve the solids. The gelatine and Sucralose were then incorporated into the syrup mixture using high shear and the temperature was reduced to approximately 50-60° C. Finally, the creatine mixture was added, together with the colour and flavour additives, and blended. The delivery system was moulded using standard techniques.

### 2.10 Ingredient % by Weight

| Ingredient | % by Weight |
|---|---|
| Glycerol | 30.19% |
| Propylene glycol | 2.09% |
| Functional ingredient | 3.33% |
| Bioavailability Enhancer (Gelucire | 3.33% |
| 44/14) | |
| 63 DE Corn syrup | 19.24% |
| High Fructose Corn Syrup | 22.56% |
| Gelatine | 8.58% |
| Pectin | 0.31% |
| KOH | 0.26% |
| Sweetening agents | 0.12% |
| Modified Starch | 1.92% |
| Flavour | 0.18% |
| Colour | 0.35% |
| Water | 7.53% |
| Total: | 100.00% |

The above formulations comprising a bioavailability enhancer was prepared as follows. The glycerol and propylene glycol were blended and the functional ingredient dispersed therein and the blend warmed to 40-55° C. The sugar syrups were blended with the water and warmed to 60-70° C. The gelatine, pectin, sweetening agents and other dry ingredients were preblended and introduced into the syrup under shear. The functional ingredient blend was then uniformly blended with the gelatine preparation. Flavour and colour were then added and the whole maintained between 40° C. and 55° C.

### Example 3

### Accelerated Shelf-Life Determination

An accelerated shelf life test was conducted on the creatine delivery system prepared by the method described in Example 2.6. Microbial analysis was conducted using approved methods as described in The Compendium of Analytical Methods: HPB Methods for the Microbiological Analysis of Foods (Volume 2) issued by the Health Products and Food Branch of Health Canada. After subjecting samples of the delivery system to a temperature of 35° C. and a relative humidity of 45-55% for a period of 35 days, the samples were tested for the presence of various microorganisms as listed in Table 3. The average water activity of the samples tested was approximately 0.51.

The results, as shown in Table 3, indicate that after a period of 35 days at the above-described conditions, microbial contamination was minimal and well below accepted levels. Based on these results, the delivery system is shown to have a stable shelf life of at least one year from the date of manufacture.

In addition to the above microbial analysis, the creatine level in each sample was determined by HPLC prior to the test and after 35 days. The average creatine content for four samples randomly selected for analysis after 35 days was compared to the average creatine content for three samples taken prior to the shelf life test. The results indicated that levels of creatine monohydrate remained stable in the jujubes after 35 days exposure to the above-described conditions. Prior to the start of the experiment, three jujubes had an average of 13.4% by weight of creatine monohydrate. After 35 days, four jujubes were shown to have an average of 14.2% by weight of creatine monohydrate, which is within the error limits of the analysis performed.

TABLE 3
Microbial Analysis of a Creatine Delivery System - Accelerated Shelf
Life Determination
Water activity: approximately 0.51
Time: 35 days
Temperature: 35° C.
Humidity: 45-55%

| | HPB REFERENCE | RESULTS (No. |
|---|---|---|
| TEST CONDUCTED | NUMBER | Colonies/gm product) |
| Total aerobic plate count | MFHPB-18 | <10 |
| Total coliforms | MFHPB-34 | <10 |
| E. coli | MFHPB-34 | <10 |
| Yeast | MFHPB-22 | <50 |
| Mould | MFHPB-22 | <50 |
| Yeast Osmophilic | MFHPB-22 | <50 |
| Mould Osmophilic | MFHPB-22 | <50 |
| Staphylococcus aureus | MFHPB-21 | <25 |
| Salmonella | MFHPB-20 | not detected |

### Example 4

### Analysis of Water Activity of the Delivery System

Water activity was measured in samples of the delivery system that had been prepared according to the method described in Example 2.6.

The procedure for measuring water activity is based on the fact that the water activity of a sample is equal to the relative humidity created by the sample in a closed environment when in equilibrium. The procedure uses a water activity meter constructed by David Brookman & Associates (DB&A). The DB&A Water Activity Meter uses an Omega Engineering HX92C Relative Humidity indicator to measure the relative humidity within a closed environment containing the sample. The Omega probe converts the relative humidity (R.H.) into milliamperes (ma), where 4 ma equals 0% R.H. and 20 ma equals 100% R.H. The water activity meter is calibrated to 11.3% R.H. using a saturated solution of LiCl and to 75.3% R.H. using a saturated solution of NaCl.

The samples are manually macerated in a plastic bag and then transferred to a 30 ml sample bottle. The bottles are filled with sample to at least 1 cm from the shoulder. The bottles are capped until use and stored at room temperature. Measurements are taken by screwing the sample bottle onto the DB&A meter probe and the bottle probe assembly is maintained in a vertical position in a rack. Measurements are taken at hourly intervals at room temperature (20-22° C.) until such time that successive readings do not vary more than 1%.

Random sampling of the jujubes was conducted. The water activity (a_{w}) was determined to be 0.507, 0.515 and 0.544. These values are well below levels those that favour the growth of microorganisms. It has been shown that microorganisms generally grow best between a_{w} values of 0.995-0.980 and most microbes will cease to grow at a_{w} values less than 0.900.

### Example 5

### In Vivo Testing I

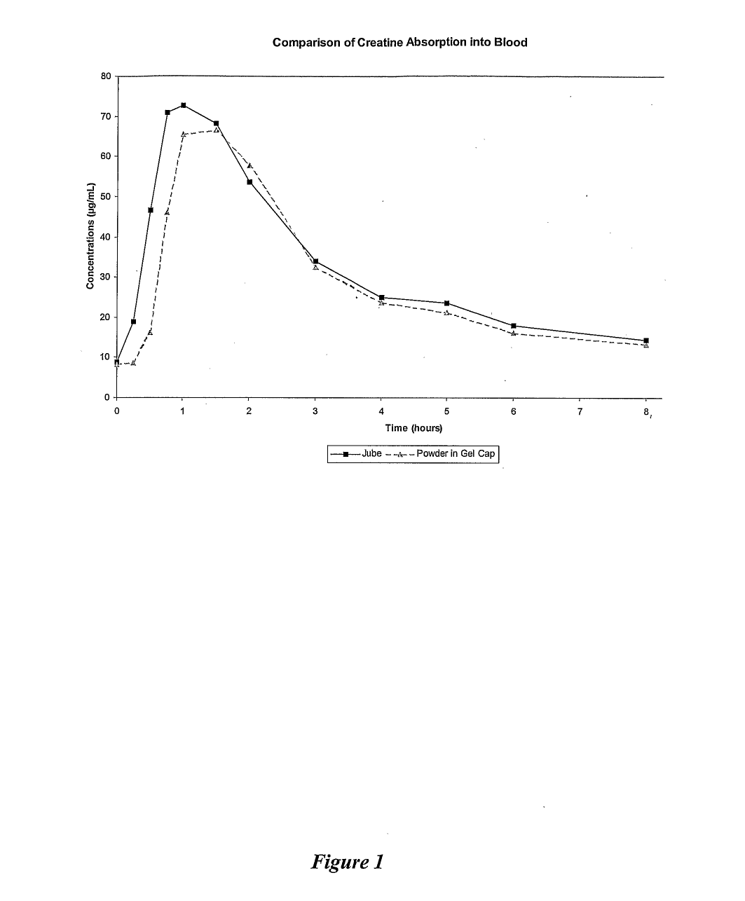

The following example demonstrates the uptake of a functional ingredient (creatine) into the blood after consumption of a delivery system formulated with a matrix as described herein. Serum concentration levels of creatine of subjects who ingested either 3.5 gram of micronized creatine powder in capsule format or 3.5 gram of micronized creatine in jujubes (prepared as described in Example 2.5) were analysed by mass spectroscopy. Seven individuals were enrolled in the test, with an age range between 18 and 50 years. Individuals fasted overnight prior to administration of the creatine. The test protocol was as follows. Individuals were administered jujube containing 3.5 g creatine with 8 oz water. Blood samples were taken every 15 minutes for the first hour, every 30 minutes for the second hour and subsequently at hourly intervals for a total of 8 hours after administration. After sufficient period of time to allow blood creatine levels to return to normal, the subjects were administered 5 capsules containing a total of 3.5 g creatine with 8 oz water. Blood samples were taken at the same time intervals as indicated above. Results are shown in FIG. 1.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

The embodiments of the invention in which an exclusive property or privilege is claimed are defined as follows:

1. An oral gel delivery system for calcium comprising one or more sources of calcium substantially uniformly dispersed in a gel matrix, said delivery system having a final moisture content of between about 10% and about 40% by weight and a water activity of less than about 0.9, and said gel matrix comprising:
a) one or more hydrocolloids;
b) one or more sugars, sugar syrups, sugar alcohols, or a combination thereof; and
c) one or more polyhydric alcohols.

2. The oral gel delivery system according to claim 1, wherein said delivery system has a final pH between about 5.0 and about 9.0.

3. The oral gel delivery system according to claim 1 or 2, wherein said delivery system comprises up to about 40% by weight of said one or more sources of calcium.

4. The oral gel delivery system according to any one of claims 1, 2 or 3, wherein said delivery system comprises between about 0.1% and about 17% by weight of said one or more hydrocolloids, between about 15% and about 55% by weight of said one or more sugars, sugar syrups, sugar alcohols, or combination thereof, and between about 5% and about 50% by weight of said one or more polyhydric alcohols.

5. The oral gel delivery system according to any one of claims 1, 2, 3 or 4, wherein said one or more hydrocolloids are selected from the group of: gelatine, gellan, pectin, modified starch, cellulose and modified cellulose.

6. The oral gel delivery system according to any one of claims 1, 2, 3, 4 or 5, wherein said one or more sugars, sugar syrups or sugar alcohols are selected from the group of: corn syrup, high fructose corn syrup, maltitol syrup and isomalt syrup.

7. The oral gel delivery system according to any one of claims 1, 2, 3, 4, 5 or 6, wherein one or more polyhydric alcohols are selected from the group of: glycerol, lower alkyl ester derivatives of glycerol, propylene glycol and short chain polyalkylene glycols.

8. The oral gel delivery system according to any one of claims 1, 2, 3, 4, 5, 6 or 7 further comprising one or more functional ingredients, wherein the total amount of said one or more sources of calcium and said one or more functional ingredients is less than or equal to 40% by weight of said delivery system.

9. The oral gel delivery system according to claim 8, wherein said one or more functional ingredient is selected from the group of: inulin, fructooligosaccharides, Vitamin D, Vitamin K, Vitamin C, magnesium, phosphorus, zinc, copper, boron, manganese, selenium, fluoride and isoflavones.

10. The oral gel delivery system according to claim 8, wherein said one or more functional ingredients are selected from the group of: fructooligosaccharides, Vitamin D, magnesium, isoflavones, and combinations thereof.

11. An oral gel delivery system for calcium comprising one or more sources of calcium substantially uniformly dispersed in a gel matrix, said delivery system having a final moisture content of between about 10% and about 30% by weight and a water activity of less than about 0.7, and said gel matrix comprising:
a) one or more hydrocolloids selected from the group of: modified starch, gelatine, gellan, pectin, cellulose and modified cellulose;
b) one or more sugar syrups selected from the group of: corn syrup, high fructose corn syrup, maltitol syrup and isomalt syrup, and
c) one or more polyhydric alcohols selected from the group of: glycerol and propylene glycol.

12. The oral gel delivery system according to claim 11, wherein said delivery system has a final pH between about 6.0 and about 8.5.

13. The oral gel delivery system according to claim 11 or 12, wherein said delivery system comprises up to about 40% by weight of said one or more sources of calcium.

14. The oral gel delivery system according to any one of claims 11, 12 or 13, wherein said delivery system comprises between about 0.1% and about 17% by weight of said one or more hydrocolloids, between about 15% and about 55% by weight of said one or more sugar syrups, and between about 5% and about 50% by weight of said one or more polyhydric alcohols.

15. The oral gel delivery system according to any one of claims 11, 12, 13 or 14 further comprising one or more functional ingredients, wherein the total amount of said one or more sources of calcium and said one or more functional ingredients is less than or equal to 40% by weight of said delivery system

16. The oral gel delivery system according to claim 15, wherein said one or more functional ingredients is selected from the group of: inulin, fructooligosaccharides, Vitamin D, Vitamin K, Vitamin C, magnesium, phosphorus, zinc, copper, boron, manganese, selenium, fluoride and isoflavones.

17. Use of a gel matrix comprising:
a) one or more hydrocolloids;
b) one or more sugars, sugar syrups, sugar alcohols, or a combination thereof, and
c) one or more polyhydric alcohols
in the preparation of an oral gel delivery system for calcium, wherein said delivery system comprises one or more sources of calcium substantially uniformly dispersed in said gel matrix, and said delivery system has a final moisture content of between about 10% and about 40% by weight and a water activity of less than about 0.9.

18. The use according to claim 17, wherein said delivery system comprises up to about 40% by weight of said one or more sources of calcium.

19. A process for preparing an oral gel delivery system for calcium, said process comprising the steps of:
(i) preparing a blend of one or more hydrocolloids, one or more sugars, sugar syrups, sugar alcohols, or a combination thereof, and optionally water at a temperature of less than 100° C., wherein said hydrocolloid(s), said sugars, sugar syrups and/or sugar alcohols and said water are in a ratio that will provide a final moisture content to the delivery system of between about 10% and about 40% by weight;
(ii) reducing the temperature of said blend to between about 50° C. and about 80° C.;
(iii) dispersing one or more sources of calcium in a solvent comprising one or more polyhydric alcohols at a temperature at or below about 70° C. to provide a solvent mixture;
(iv) combining said blend from step (ii) with said solvent mixture to provide a gel matrix, and
(v) moulding said gel matrix to provide said oral gel delivery system.

20. The process according to claim 19, wherein the amount of said one or more sources of calcium dispersed in said solvent in step (iii) provides up to 40% by weight of said source(s) of calcium in the final delivery system.

21. The process according to claim 19 or 20, wherein preparing said blend in step (i) is at a temperature between about 60° C. and about 80° C.

22. The process according to any one of claims 19, 20 or 21, wherein dispersing said one or more sources of calcium in said solvent in step (iii) is at a temperature below about 50° C.

23. An oral gel delivery system for calcium prepared by the process of any one of claims 19, 20, 21 or 22.

24. Use of an effective amount of the oral gel delivery system according to any one of claims 1, 2, 3, 4, 5, 6, 7, 8, 9 or 10 as a calcium supplement for an animal in need thereof.

25. The use according to claim 24, wherein said to calcium supplement is for preventing bone loss in said animal.

26. The use according to claim 24, wherein said to calcium supplement is for maintaining or increasing bone strength in said animal.

27. The use according to any one of claims 24, 25 or 26, wherein said animal is a human.

28. Use of an effective amount of the oral gel delivery system according to any one of claims 11, 12, 13, 14, 15 or 16 as a calcium supplement for an animal in need thereof.

29. The use according to claim 28, wherein said to calcium supplement is for preventing bone loss in said animal.

30. The use according to claim 28, wherein said to calcium supplement is for maintaining or increasing bone strength in said animal.

31. The use according to any one of claims 28, 29 or 30, wherein said animal is a human.

32. A kit for the delivery of calcium to an animal comprising one or more units of the oral gel delivery system according to any one of claims 1, 2, 3, 4, 5, 6, 7, 8, 9 or 10 and optionally instructions for use.

33. A kit for the delivery of calcium to an animal comprising one or more units of the oral gel delivery system according to any one of claims 11, 12, 13, 14, 15 or 16 and optionally instructions for use.

34. The kit according to claim 32 or 33, wherein said animal is a human.
